(19) United States
(12) Patent Application Publication
(10) Pub. No.: US 2006/0130159 A1
(43) Pub. Date: Jun. 15, 2006

(54) Method of purifying recombinant MSP 1-42 derived from Plasmodium falciparum

(76) Inventors: Nick Masiello, Uxbridge, MA (US); Mark Perreault, Leominster, MA (US)

Correspondence Address: GTC BIOTHERAPEUTICS, INC., 175 CROSSING BOULEVARD, SUITE 410, FRAMINGHAM, MA, 01702, US

(21) Appl. No.: 11/007,852

(22) Filed: Dec. 9, 2004

(51) Int. Cl. A01K 67/027; C07K 14/445

(52) U.S. Cl. 800/7; 530/350

(57) ABSTRACT

Methods of purifying MSP-1 from a variety of source materials including from the milk of transgenic mammals.

### FIELD OF THE INVENTION

The invention relates to the production and processing of a heterologous gene expression products. More particularly, the invention relates to the expression of malaria genes in higher eukaryote cell systems and the purification of these recombinant protein products for therapeutic use.

### BACKGROUND OF THE INVENTION

As stated above, the present invention relates generally to the production of recombinant proteins by higher eukaryotic cells or cell systems. In particular, preferred embodiments of the current invention rely upon the production of the parasite protein of interest in the milk of transgenic mammals. The current invention provides improved methods for processing-out or purifying recombinant proteins of interest from a given feedstream. The ultimate goal of the current invention is to provide methods useful in the production of a vaccine for therapeutically treating malaria and related pathological conditions.

Recombinant production of certain heterologous gene products is often difficult in in vitro cell culture systems or in vivo recombinant production systems. For example, many researchers have found it difficult to express proteins derived from bacteria, parasites or virus genomes in cell culture systems different from the cell from which the protein was originally derived. This problem is particularly acute when the prokaryotic or protozoan protein of interest is expressed in mammalian cell culture systems. One example of a therapeutically important protein which has been difficult to produce by mammalian cell culture is the malaria merozoite surface protein (MSP-1). This protein sequence is conserved in parasites and has long been thought a promising protein target for a vaccine.

Malaria is a serious heath problem in tropical countries. Resistance to existing drugs is fast developing and a vaccine is urgently needed. Of the number of antigens that get expressed during the life cycle of Plasmodium falciparum, MSP-1 is the most extensively studied and promises to be the most successful candidate with which to develop an effective vaccination protocol. Individuals exposed to Plasmodium falciparum develop antibodies against MSP-1, and studies have shown that there is a correlation between a naturally acquired immune response to MSP-1 and a reduction in malaria morbidity and/or severity. In a number of studies, immunization with purified native MSP-1 or recombinant fragments of the protein has induced at least partial protection from the parasite (Diggs et al, (1993) PARASITOL TODAY 9:300-02). Thus, MSP-1 is an important target for the development of a vaccine against Plasmodium falciparum. The MSP-1 protein is itself a 190-220 kDA glycoprotein. The C-terminal region has been the focus of recombinant production for use as a vaccine. However, a major problem in developing MSP-1 as a vaccine is the difficulty in obtaining recombinant proteins in bacterial or yeast expression systems that are equivalent in immunological potency and/or secondary structure to the affinity purified native protein (Chang et al., (1992) J. IMMUNOL. 148:548-555). Another substantial difficulty is in producing enough of the protein to make vaccine availability feasible on a medically meaningful scale. Improved procedures for enhancing expression of sufficient quantities of MSP-1 would be advantageous. Also needed are methods of processing and purifying the physiologically active molecule from the bioreactor producing it at high volume.

As indicated above, once production of the protein is accomplished the next hurdle to be overcome is the processing and purification of the physiologically potent protein in good yield. Without this and no widely available vaccine would be possible. With improvements in the production of exogenous proteins or other molecules of interest from biological systems there has been increasing pressure on industry to develop new techniques to enhance and make more efficient the purification and recovery processes for the biologics and pharmaceuticals so produced. That is, with an increased pipeline of new products, there is substantial interest in devising methods to bring these therapeutics, in commercial volumes, to market quickly. At the same time the industry is facing new challenges in terms of developing novel processes for the recovery of transgenic proteins and antibodies from various bodily fluids including milk and urine. The larger the scale of production the more complex these problems often become. In addition, there are further challenges imposed in terms of meeting product purity and safety, notably in terms of virus safety and residual contaminants, such as DNA and host cell proteins that might be required to be met by the various governmental agencies that oversee the production of biologically useful pharmaceuticals.

Several methods are currently available to separate molecules of biological interest, such as the P. falciparum protein MSP-1, from mixtures thereof. One important such technique is affinity chromatography, which separates molecules on the basis of specific and selective binding of the desired molecules to an affinity matrix or gel, while the undesirable molecule remains unbound and can then be moved out of the system. Affinity gels typically consist of a ligand-binding moiety immobilized on a gel support. For example, GB 2,178,742 utilizes an affinity chromatography method to purify hemoglobin and its chemically modified derivatives based on the fact that native hemoglobin binds specifically to a specific family of poly-anionic moieties. For capture these moieties are immobilized on the gel itself. In this process, unmodified hemoglobin is retained by the affinity gel, while modified hemoglobin, which cannot bind to the gel because its poly-anion binding site is covalently occupied by the modifying agent, is removed from the system. Affinity chromatography columns are highly specific and thus yield very pure products; however, affinity chromatography is a relatively expensive process and therefore very difficult to put in place for commercial operations.

Typically, genetically engineered biopharmaceuticals are purified from a supernatant containing a variety of diverse host cell contaminants. Reversed-phase high-performance liquid chromatography (RP-HPLC) can be used for protein purification because it can efficiently separate molecular species that are exceptionally similar to one another in terms of structure or weight. Procedures utilizing RP-HPLC have been published for many molecules. McDonald and Bidlingmeyer, “Strategies for Successful Preparative Liquid Chromatography”, in PREPARATIVE LIQUID CHROMATOGRAPHY, Brian A. Bidlingmeyer (New York: Elsevier Science Publishing, 1987), vol. 38, pp. 1-104; Lee et al., PREPARATIVE HPLC. 8th Biotechnology Symposium, Pt. 1, 593-610 (1988).

The use of membranes in the recovery processes for molecular products at industrial scale, and the associated use of many types of membranes and membrane techniques is also known. In these methods the essential feature is that particles, suspended in a liquid feedstream are separated on the basis of their size. In the simplest form of this process, a solution is forced under pressure through a filter membrane with pores of a defined size. Particles larger than the pore size of the membrane filter are retained, while smaller solutes are carried convectively through the membrane with the solvent. Such membrane filtration processes generally falls within the categories of reverse osmosis, ultrafiltration, and microfiltration, depending on the pore size of the membrane.

It is also important to mention that membrane filtration as a separation technique is widely used in the biotechnology field. Depending on membrane type, it can be classified as microfiltration or ultrafiltration. Microfiltration membranes, with a pore size between 0.1 and 10 μm, are typically used for clarification, sterilization, removal of microparticulates, or for cell harvests. Ultrafiltration membranes, with much smaller pore sizes between 0.001 and 0.1 μm, are used for separating out and concentrating dissolved molecules (protein, peptides, nucleic acids, carbohydrates, and other biomolecules), for exchange buffers, and for gross fractionation.

The methods of the current invention also provide precise combinations of filters and conditions that allow the optimization of the yield of molecules of interest (ex: MSP-1) from a given feedstream. In these methods important the process parameters such as pH and temperature are precisely manipulated.

Accordingly, the methods and processes of the current invention provide for the purification of sufficient quantities of the MSP-1 molecule to allow the production of a vaccine that will positively impact the treatment of malaria. This may be accomplished, according to the current invention, through the use of physiologically active recombinant MSP-1 produced by a higher eukaryote cell or transgenic animal and then processed out of the feedstream of the originating bioreactor in therapeutically effective amounts.

### SUMMARY OF THE INVENTION

Briefly stated, the present invention relates generally to the production and purification of MSP-1 in a state that makes it available for therapeutic use as a basis of a vaccine for malaria. This invention is also directed to compositions of purity and form that are suitable for pharmaceutical use and treatment of medical conditions.

The present invention provides improved recombinant DNA compositions, procedures, and purification methods for increasing the mRNA levels and protein expression of physiologically active malarial surface antigen MSP-1 in cell culture systems, mammalian cell culture systems, or in transgenic mammals. The preferred protein candidate for expression in an expression system in accordance with the invention is a C-terminal derivative of MSP-1 having a DNA coding sequence with reduced AT content, and eliminated mRNA instability motifs and rare codons relative to the recombinant expression systems. Thus, in a first aspect, the invention provides a DNA sequence derived from the sequence shown in SEQ ID NO 2. This derivative sequence is shown in SEQ ID NO 1. Another objective of the current invention provides a process for preparing a modified nucleic acid of the invention comprising the steps of lowering the overall AT content of the natural gene encoding MSP-1, eliminating all mRNA instability motifs and replacing all rare codons with a preferred codon of the mammary gland tissue, all by replacing specific codons in the natural gene with codons recognizable to, and preferably preferred by mammary gland tissue and which code for the same amino acids as the replaced codon. This aspect of the invention further includes modified nucleic acids prepared according to the process of the invention.

In a third aspect, the invention also provides vectors comprising modified MSP-1 nucleic acids of the invention and a goat beta casein promoter and signal sequence, and host cells transformed with nucleic acids of the invention.

In a fourth aspect, the invention provides transgenic non-human mammals whose germlines comprise a nucleic acid of the invention.

In a fifth aspect, the invention provides a DNA vaccine comprising a modified MSP-1 gene according to the processing and purification methods of the invention.

It is an object of the present invention to provide tangential-flow filtration processes for separating species such as particles and molecules by size, which processes are selective for the species of interest, resulting in higher-fold purification thereof.

It is another object to provide improved filtration processes, including ultrafiltration processes, for separating biological macromolecules such as the MSP-1 protein which processes minimize concentration polarization and do not increase flux.

It is another object to provide a filtration process that can separate by size species that are less than ten-fold different in size and do not require dilution of the mixture prior to filtration. These and other objects will become apparent to those skilled in the art. Other features and advantages of this invention will become apparent in the following detailed description of preferred embodiments of this invention, taken with reference to the accompanying drawings. The biologics industry is becoming increasingly concerned with product safety and purity as well as cost of goods. The use of tangential flow filtration (TFF), according to the current invention, is a rapid and more efficient method for biomolecule separation. It can be applied to a wide range of biological fields such as immunology, protein chemistry, molecular biology, biochemistry, and microbiology.

These and other objects which will be more readily apparent upon reading the following disclosure may be achieved by the present invention.

### BRIEF DESCRIPTION OF THE DRAWINGS

FIG. 1 Shows the cDNA sequence of MSP1-42 modified in accordance with the invention [SEQ ID NO 1] in which 306 nucleotide positions have been replaced to lower AT content and eliminate mRNA instability motifs while maintaining the same protein amino acid sequence of MSP1-42. The large letters indicate nucleotide substitutions.

FIG. 2 Shows the nucleotide sequence coding sequence of the “wild type” or native MSP1-42 [SEQ ID NO 2].

FIGS. 3A and 3B Show a codon usage table for wild type MSP-142 (designated “MSP wt” in the table) and the new modified MSP1-42 gene (designated “edited MSP” in the table) and several milk protein genes (casein genes derived from goats and mouse). The numbers in each column indicate the actual number of times a specific codon appears in each of the listed genes. The new MSP1-42 synthetic gene was derived from the mammary specific codon usage by first choosing GC rich codons for a given amino acid combined with selecting the amino acids used most frequently in the milk proteins.

FIG. 4A-4C Show MSP1-42 constructs GTC 479, GTC 564, and GTC 627, respectively as are described in the examples.

FIG. 5A Shows a Northern analysis wherein construct GTC627 comprises the new MSP-142 gene modified in accordance with the invention, GTC479 is the construct comprising the native MSP-142 gene, and construct GTC469 is a negative control DNA.

FIG. 5B Shows a Western analysis wherein the eluted fractions after affinity purifications numbers are collected fractions. The results show that fractions from GTC679 the modified MSP-142 synthetic gene construct reacted with polyclonal antibodies to MSP-1 and the negative control GTC479 did not.

FIG. 6 Shows the nucleic acid sequences of OT1 [SEQ ID NO 3], OT2 [SEQ ID NO 4], MSP-8 [SEQ ID ON 5], MSP-2 [SEQ ID NO 6] and MSPI [SEQ ID NO 7] described in the Examples.

FIG. 7 Shows a schematic representation of plasmid BC574.

FIG. 8 Shows a schematic representation of BC670.

FIG. 9 Shows a schematic representation of BC620.

FIG. 10 Shows a representation of a Western blot of MSP transgenic milk.

FIG. 11 Shows a schematic representation of the nucleotide sequence of M5P42-2 [SEQ ID NO 8].

FIG. 12 Shows a representation of a Western blot of BC-718 expression in transgenic milk.

FIG. 13 Shows a Flowchart of an Embodiment of the Process of Creating Cloned Animals through Nuclear Transfer. [001]

FIG. 14 Shows SDS-PAGE comparison of TFF processes with arginine and urea solubilization of MSP1-42.

FIG. 15 Shows the profile of an initial POROS HS50 column purification using a sodium chloride step gradient.

FIG. 16 Shows SDS-PAGE of MSP1-42 degradation observed at 4° C.

FIG. 17 Shows a degradation study using fresh natural lactation milk with and without protease inhibitors and comparing TFF and centrifugation processes (with arginine solubilization) for clarification.

FIG. 18 Shows a study looking at TFF with arginine+urea for MSP1-42 solubilization.

FIG. 19 Shows a study looking at TFF with urea only for MSP1-42 solubilization.

FIG. 20 Shows the profile of an early hydroxyapatite (CHT) column with urea that was eluted with a linear phosphate gradient.

FIG. 21 Shows the profile of an early Source 30Q column purification using a sodium chloride step gradient.

FIG. 22 Shows the profile of a reoptimized POROS HS50 column containing urea and using a higher pH step for elution.

FIG. 23 Shows the profile of a reoptimized CHT column using a phosphate step gradient for elution.

FIG. 24 Shows the profile of a reoptimized Source 30Q column containing urea and using a sodium chloride step gradient.

FIG. 25 Shows SDS-PAGE of MSP1-42 fractions through the first three steps of the purification process.

FIG. 26 Shows an early Superdex 200 column profile (the final step of the MSP1-42 purification process).

FIG. 27 Shows the full MSP-1 purification process of the invention.

### DETAILED DESCRIPTION

The following abbreviations have designated meanings in the specification:

Abbreviation Key:

pH A term used to describe the hydrogen-ion activity of a chemical or compound according to well-known scientific parameters.

PPM Parts Per Million

SDS-PAGE SDS (sodium dodecyl sufate) Poly-Acrylamide Gel electrophoresis

SEC Size Exclusion Chromatography

TFF Tangential Flow Filtration

PEG Polyethylene glycol

TMP Transmembrane Pressure

CHT Ceramic Hyrdoxyapatite

Explanation of Terms:

Biological Fluid—an aqueous solution produced by an organism, such as a mammal, bird, amphibian, or reptile, which contains proteins that are secreted by cells that are bathed in the aqueous solution. Examples include: milk, urine, saliva, seminal fluid, vaginal fluid, synovial fluid, lymph fluid, amniotic fluid, blood, sweat, and tears; as well as an aqueous solution produced by a plant, including, for example, exudates and guttation fluid, xylem, phloem, resin, and nectar.

Biological-fluid producing cell—A cell that is bathed by a biological fluid and that secretes a protein into the biological fluid.

Biopharmaceutical—shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.

Caprine—Of or relating to various species of goats.

Clarification—The removal of particulate matter from a solution so that the solution is able to pass through a 0.2 μm membrane.

Colloids—Refers to large molecules that do not pass readily across capillary walls. These compounds exert an oncotic (i.e., they attract fluid) load and are usually administered to restore intravascular volume and improve tissue perfusion.

Concentration—The removal of water and small molecules with a membrane such that the ratio of retained molecules to small molecules increases.

Concentration Polarization—The accumulation of the retained molecules (gel layer) on the surface of the membrane caused by a combination of factors: transmembrane pressure, crossflow velocity, sample viscosity, and solute concentration.

Crossflow Velocity—Velocity of the fluid across the top of the membrane surface. CF=Pi−Po where Pi is pressure at the inlet and Po is pressure at the outlet and is related to the retentate flow rate.

Diafiltration—The fractionation process of washing smaller molecules through a membrane, leaving the larger molecule of interest in the retentate. It is a convenient and efficient technique for removing or exchanging salts, removing detergents, separating free from bound molecules, removing low molecular weight materials, or rapidly changing the ionic or pH environment. The process typically employs a microfiltration membrane that is employed to remove a product of interest from a slurry while maintaining the slurry concentration as a constant.

Encoding—refers generally to the sequence information being present in a translatable form, usually operably linked to a promoter (e.g., a beta-casein or beta-lacto globulin promoter). A sequence is operably linked to a promoter when the functional promoter enhances transcription or expression of that sequence. An anti-sense strand is considered to also encode the sequence, since the same informational content is present in a readily accessible form, especially when linked to a sequence which promotes expression of the sense strand. The information is convertible using the standard, or a modified, genetic code.

Expression Vector—A genetically engineered plasmid or virus, derived from, for example, a bacteriophage, adenovirus, retrovirus, poxvirus, herpesvirus, or artificial chromosome, that is used to transfer an malaria related transgenic protein coding sequence, operably linked to a promoter, into a host cell, such that the encoded recombinant malaria related transgenic protein is expressed within the host cell.

Feedstream—The raw material or raw solution provided for a process or method and containing a protein of interest and which may also contain various contaminants including microorganisms, viruses and cell fragments.

Fractionation—The preferential separation of molecules based on a physical or chemical moiety.

Functional Proteins—Proteins which have a biological or other activity or use, similar to that seen when produced endogenously.

Transgenic Slide—A glass slide for parallel electrodes that are placed a fixed distance apart. Cell couplets are placed between the electrodes to receive an electrical current for transgenic and activation.

Homologous Sequences—refers to genetic sequences that, when compared, exhibit similarity. The standards for homology in nucleic acids are either measures for homology generally used in the art or hybridization conditions. Substantial homology in the nucleic acid context means either that the segments, or their complementary strands, when compared, are identical when optimally aligned, with appropriate nucleotide insertions or deletions, in at least about 60% of the residues, usually at least about 70%, more usually at least about 80%, preferably at least about 90%, and more preferably at least about 95 to 98% of the nucleotides. Alternatively, substantial homology exists when the segments will hybridize under selective hybridization conditions, to a strand, or its complement. Selectivity of hybridization exists when hybridization occurs which is more selective than total lack of specificity. Typically, selective hybridization will occur when there is at least about 55% homology over a stretch of at least about 14 nucleotides, preferably at least about 65%, more preferably at least about 75%, and most preferably at least about 90%.

Leader sequence or a “signal sequence”—a nucleic acid sequence that encodes a protein secretory signal, and, when operably linked to a downstream nucleic acid molecule encoding a transgenic protein and directs secretion. The leader sequence may be the native human leader sequence, an artificially-derived leader, or may obtained from the same gene as the promoter used to direct transcription of the transgene coding sequence, or from another protein that is normally secreted from a cell.

Milk-producing cell—A cell (e.g., a mammary epithelial cell) that secretes a protein into milk.

Milk-specific promoter—A promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, and κ-casein promoter; the whey acidic protein (WAP) promoter (Gorton et al., BIOTECHNOLOGY 5: 1183-1187, 1987); the β-lactoglobulin promoter (Clark et al., BIOTECHNOLOGY 7: 487-492, 1989); and the α-lactalbumin promoter (Soulier et al., FEBS LETTS. 297:13, 1992). Also included are promoters that are specifically activated in mammary tissue and are thus useful in accordance with this invention, for example, the long terminal repeat (LTR) promoter of the mouse mammary tumor virus (MMTV).

Nuclear Transfer—This refers to a method of cloning wherein the nucleus from a donor cell is transplanted into an enucleated oocyte.

Operably Linked—A gene and one or more regulatory sequences are connected in such a way as to permit gene expression when the appropriate molecules (e.g., transcriptional activator proteins) are bound to the regulatory sequences.

Ovine—Of or relating to or resembling sheep.

Pharmaceutically Pure—This refers to transgenic protein that is suitable for unequivocal biological testing as well as for appropriate administration to effect treatment of a human patient. Substantially pharmaceutically pure means at least about 90% pure.

Promoter—A minimal sequence sufficient to direct transcription. Also included in the invention are those promoter elements which are sufficient to render promoter-dependent gene expression controllable for cell type-specific, tissue-specific, temporal-specific, or inducible by external signals or agents; such elements may be located in the 5′ or 3′ or intron sequence regions of the native gene.

Tangential Flow Filtration—A process in which the fluid mixture containing the components to be separated by filtration is re-circulated at high velocities tangential to the plane of the membrane to increase the mass-transfer coefficient for back diffusion. In such filtrations a pressure differential is applied along the length of the membrane to cause the fluid and filterable solutes to flow through the filter. This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

Transmembrane Pressure—The pressure differential gradient that is applied along the length of a filtration membrane to cause fluid and filterable solutes to flow through the filter. In tangential flow systems, highest TMP's are at the inlet (beginning of flow channel) and lowest at the outlet (end of the flow channel). TMP is calculated as an average pressure of the inlet, outlet, and filtrate ports.

Transformed cell or Transfected cell—A cell (or a descendent of a cell) into which a nucleic acid molecule encoding malaria related has been introduced by means of recombinant DNA techniques. The nucleic acid molecule may be stably incorporated into the host chromosome, or may be maintained episomally.

Transgene—Any piece of a nucleic acid molecule that is inserted by artifice into a cell, or an ancestor thereof, and becomes part of the genome of the animal which develops from that cell. Such a transgene may include a gene which is partly or entirely exogenous (i.e., foreign) to the transgenic animal, or may represent a gene having identity to an endogenous gene of the animal.

Transgenic—Any cell that includes a nucleic acid molecule that has been inserted by artifice into a cell, or an ancestor thereof, and becomes part of the genome of the animal which develops from that cell.

Transgenic Organism—An organism into which genetic material from another organism has been experimentally transferred, so that the host acquires the genetic information of the transferred genes in its chromosomes in addition to that already in its genetic complement.

Vector—As used herein means a plasmid, a phage DNA, or other DNA sequence that (1) is able to replicate in a host cell, (2) is able to transform a host cell, and (3) contains a marker suitable for identifying transformed cells.

According to the present invention, there is provided methods for the production and processing of a transgenic protein of interest, the process comprising expressing in the milk of a transgenic non-human placental mammal a transgenic protein useful in the treatment of malaria or related pathologies and processing a useful form of the protein therefrom. The term “treating”, “treat” or “treatment” as used herein includes preventative (e.g., prophylactic) and palliative treatment.

To recombinantly produce a protein of interest a nucleic acid encoding a transgenic protein can be introduced into a host cell, e.g., a cell of a primary or immortalized cell line. The recombinant cells can be used to produce the transgenic protein, including a cell surface receptor that can be secreted from a mammary epithelial cell. A nucleic acid encoding a transgenic protein can be introduced into a host cell, e.g., by homologous recombination. In most cases, a nucleic acid encoding the transgenic protein of interest is incorporated into a recombinant expression vector.

The nucleotide sequence encoding a transgenic protein can be operatively linked to one or more regulatory sequences, selected on the basis of the host cells to be used for expression. The term “operably linked” means that the sequences encoding the transgenic protein compound are linked to the regulatory sequence(s) in a manner that allows for expression of the transgenic protein. The term “regulatory sequence” refers to promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990), the contents of which are incorporated herein by reference.

Regulatory sequences include those that direct constitutive expression of a nucleotide sequence in many types of host cells, those that direct expression of the nucleotide sequence only in certain host cells (e.g., tissue-specific regulatory sequences) and those that direct expression in a regulatable manner (e.g., only in the presence of an inducing agent). It will be appreciated by those skilled in the art that the design of the expression vector may depend on such factors as the choice of the host cell to be transformed, the level of expression of transgenic protein desired, and the like. The transgenic protein expression vectors can be introduced into host cells to thereby produce transgenic proteins encoded by nucleic acids.

Examples of mammalian expression vectors include pCDM8 (Seed et al., (1987) NATURE 3:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and SV40.

In addition to the regulatory control sequences discussed above, the recombinant expression vector can contain additional nucleotide sequences. For example, the recombinant expression vector may encode a selectable marker gene to identify host cells that have incorporated the vector. Moreover, to facilitate secretion of the transgenic protein from a host cell, in particular mammalian host cells, the recombinant expression vector can encode a signal sequence operatively linked to sequences encoding the amino-terminus of the transgenic protein such that upon expression, the transgenic protein is synthesized with the signal sequence fused to its amino terminus. This signal sequence directs the transgenic protein into the secretory pathway of the cell and is then cleaved, allowing for release of the mature transgenic protein (i.e., the transgenic protein without the signal sequence) from the host cell. Use of a signal sequence to facilitate secretion of proteins or peptides from mammalian host cells is known in the art.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, microinjection and viral-mediated transfection. Suitable methods for transforming or transfecting host cells can be found in Sambrook et al. (Molecular Cloning: A Laboratory Manual, 2^{nd} Edition, Cold Spring Harbor Laboratory press (1989)), and other laboratory manuals.

MSP 1-42

The present invention provides improved recombinant DNA compositions and procedures for increasing the mRNA levels and protein expression of the malarial surface antigen MSP-1 in cell culture systems, mammalian cell culture systems, or in transgenic mammals. The preferred protein candidate for expression in an expression system in accordance with the invention is a C-terminal derivative of MSP-1 having a DNA coding sequence with reduced AT content, and eliminated mRNA instability motifs and rare codons relative to the recombinant expression systems. Thus, in a first aspect, the invention provides a DNA sequence derived from the sequence shown in SEQ ID NO 1 This derivative sequence is shown in SEQ ID NO 1.

In preferred embodiments, the nucleic acid of the invention is capable of expressing MSP-1 in mammalian cell culture systems. As used herein, the term “expression” is meant mRNA transcription resulting in protein expression. Expression may be measured by a number of techniques known in the art including using an antibody specific for the protein of interest. By “natural gene” or “native gene” is meant the gene sequence, or fragments thereof (including naturally occurring allelic variations), which encode the wild type form of MSP-1 and from which the modified nucleic acid is derived. A “preferred codon” means a codon which is used more prevalently by the cell or tissue type in which the modified MSP-1 gene is to be expressed, for example, in mammary tissue. Not all codon changes described herein are changes to a preferred codon, so long as the codon replacement is a codon which is at least recognized by the mouse mammary tissue.

The term “reduced AT content” as used herein means having a lower overall percentage of nucleotides having A (adenine) or T (thymine) bases relative to the natural MSP-1 gene due to replacement of the A or I containing nucleotide positions or A and/or T containing codons with nucleotides or codons recognized by mouse mammary tissue and which do not change the amino acid sequence of the target protein.

In a second aspect, the invention provides a process for eliminating all mRNA instability motifs and replacing all rare codons with a preferred codon of mammary gland tissue, all by replacing specific codons in the natural gene with codons recognizable to, and preferably preferred by mammary gland tissue and which code for the same amino acids as the replaced codon. Standard reference works describing the general principals of recombinant DNA technology include Watson, J. D. et al, MOLECULAR BIOLOGY OF THE GENE, Volumes I and II the Benjamin/Cummings Publishing Company, Inc. publisher, Menlo Park, Calif. (1987) Darnell, J. E. et al., Molecular Cell Biology, Scientific American Books, Inc., Publisher, New York, N.Y. (1986); Old, R. W., et al., PRINCIPLES OF GENE MANIPULATION: AN INTRODUCTION TO GENETIC ENGINEERING, 2^{nd} edition, University of California Press, publisher, Berkeley Calif. (1981); Maniatis, T., et al., MOLECULAR CLONING: A LABORATORY MANUAL, 2^{nd} ed. Cold Spring Harbor Laboratory, publisher, Cold Spring Harbor, N.Y. (1989) and CURRENT PROTOCOLS IN MOLECULAR BIOLOGY, Ausubel et al., Wiley Press, New York, N.Y. (1992). This aspect of the invention further includes modified nucleic acids prepared according to the process of the invention. Without being limited to any theory, previous research has indicated that a conserved AU sequence (AUUUA) from the 3′ untranslated region of GM-CSF mRNA mediates selective mRNA degradation (Shaw, G. and Kamen, CELL 46:659-667). The focus in the past has been on the presence of these instability motifs in the untranslated region of a gene. The instant invention is the first to recognize an advantage to eliminating the instability sequences in the coding region of the MSP-1 gene.

In a third aspect, the invention also provides for the processing and/or purification of a MSP-1 protein of the invention from a milk feedstream derived from a transgenic animal(s).

The mammary gland expression system has the advantages of high expression levels, low cost, correct processing and accessibility. Known proteins, such as bovine and human alpha-lactalbumin have been produced in lactating transgenic animals by several researchers. (Wright et al, BIO/TECHNOLOGY 9:830-834 (1991); Vilotte et al, DIR. J. BIOCHEM.,186:43-48 (1989); Hochi et al., MOL REPROD. AND DEVEL. 33:160-164 (1992); Soulier et al., FEBS LETTERS 297(1,2):13-18 (1992)) and the system has been shown to produce high levels of protein.

In a fifth aspect, the invention provides a DNA vaccine comprising a modified MSP-1 gene according to the invention. Such DNA vaccines may be delivered without encapsulation, or they may be delivered as part of a liposome, or as part of a viral genome. Generally, such vaccines are delivered in an amount sufficient to allow expression of the modified MSP-1 gene and to elicit an antibody response in an animal, including a human, which receives the DNA vaccine. Subsequent deliveries, at least one week after the first delivery, may be used to enhance the antibody response. Preferred delivery routes include introduction via mucosal membranes, as well as parenteral administration.

### EXAMPLE 1

### Creation of a Novel Modified MSP1-42 Gene

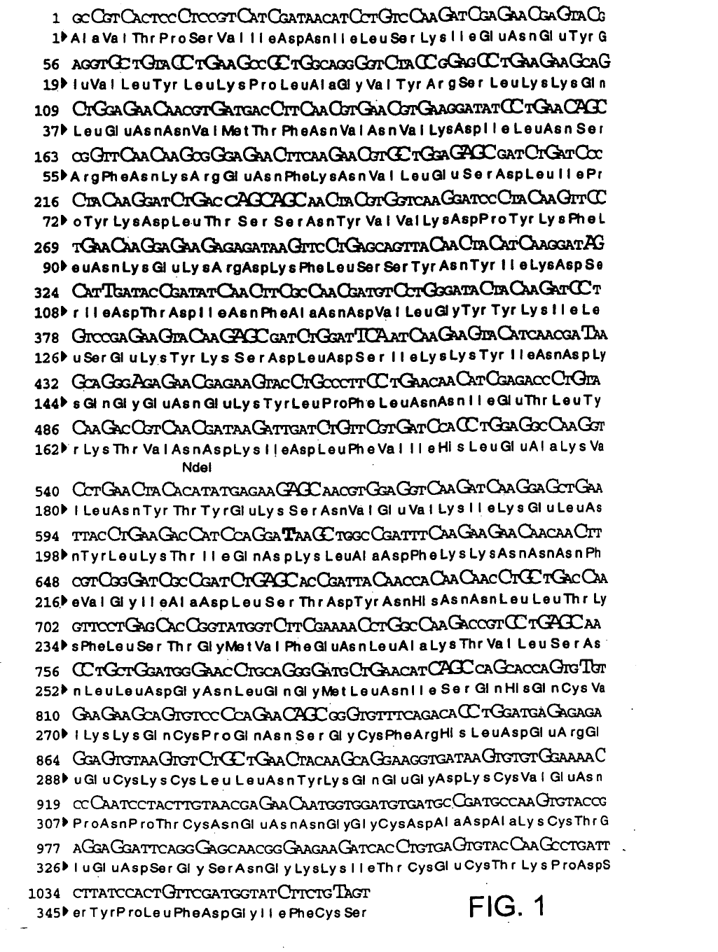

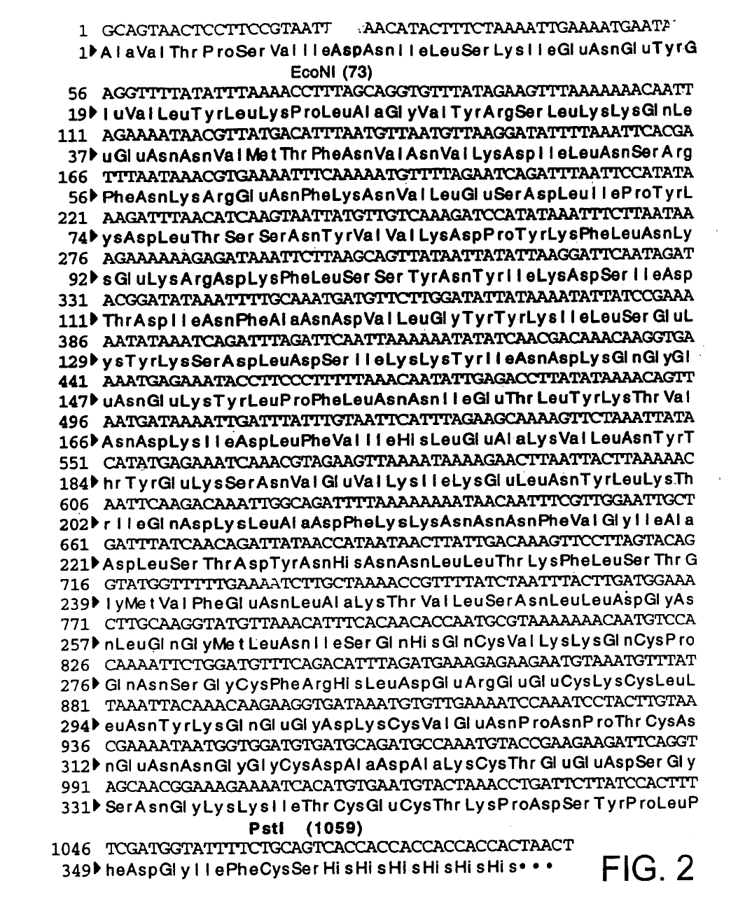

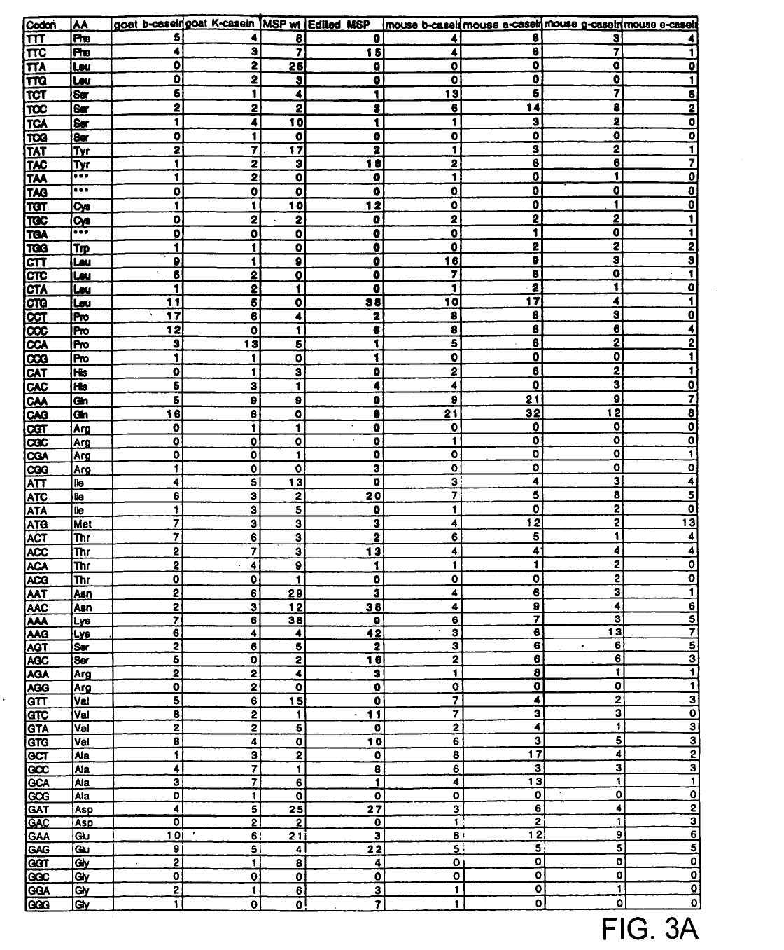

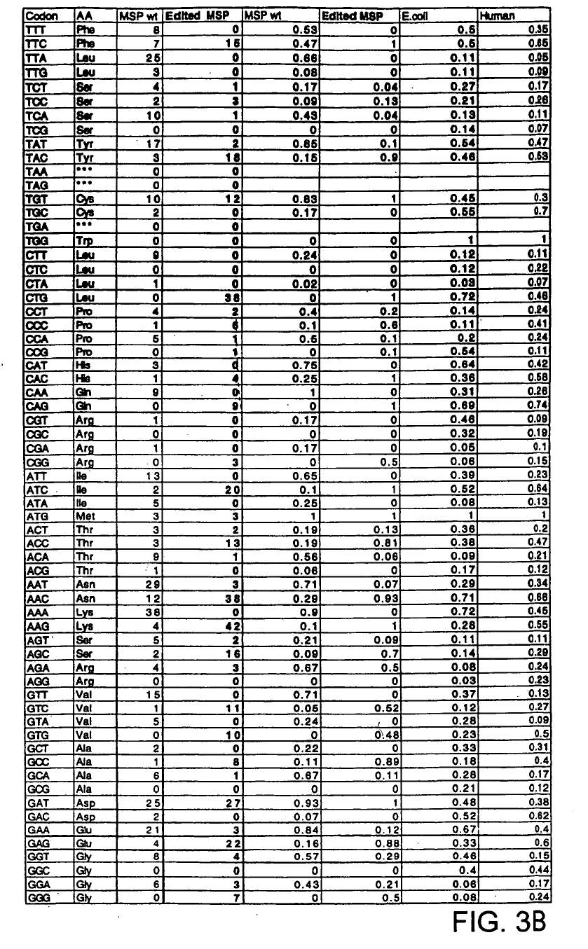

A novel modified nucleic acid encoding the C-terminal fragment of MSP-1 is provided. The novel, modified nucleic acid of the invention encoding a 42 kD C-terminal part of MSP-1 (MSP-1p) capable of expression in mammalian cells of the invention is shown in FIG. 1. The natural MSP-1₄₂ gene (FIG. 2) was not capable of being expressed in mammalian cell culture or in transgenic mice Analysis of the natural MSP-142 gene suggested several characteristics that distinguish it from mammalian genes. First, it has a very high overall AT content of 76% Second, the mRNA instability motif, AUUUA, occurred 10 times in this 1100 bp DNA segment (FIG. 2). To address these differences a new MSP-1₄₂ gene was designed. Silent nucleotide substitution was introduced into the native MSP-1₄₂ gene at 306 positions to reduce the overall AT content to 49.7%. Each of the 10 AUUUA mRNA instability motifs in the natural gene were eliminated by changes in codon usage as well. To change the codon usage, a mammary tissue specific codon usage table, FIG. 3a, was created by using several mouse and goat mammary specific proteins. The table was used to guide the choice of codon usage for the modified MSP-1₄₂ gene as described above. For example as shown in the Table in FIG. 3a, in the natural gene, 65% (25/38) of the Leu was encoded by TTA, a rare codon in the mammary gland. In the modified MSP-1₄₂ gene, 100″/˜ of the Leu was encoded by CTG, a preferred codon for Leu in the mammary gland.

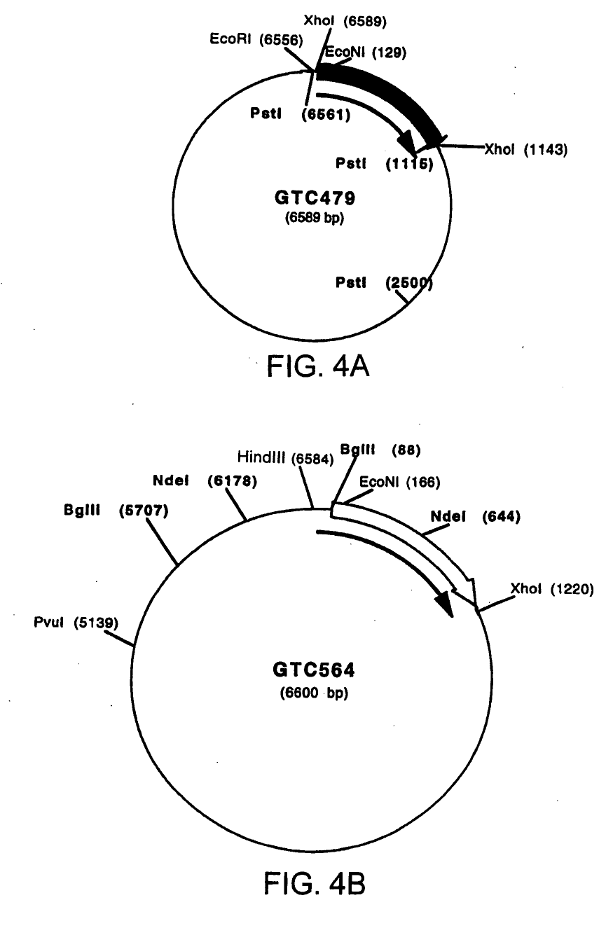

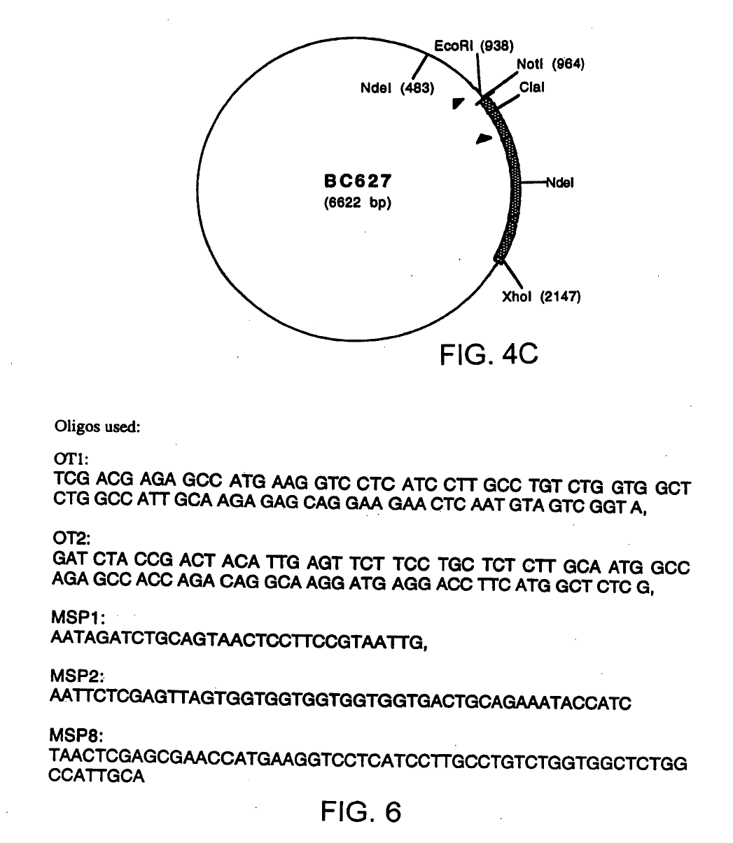

An expression vector was created using the modified MSP-1₄₂gene by fusing the first 26 amino acids of goat beta-casein to the N-terminal of the modified MSP-1₄₂ gene and a SalI-Xho I fragment which carries the fusion gene was subcloned into the XhoI site of the expression vector pCDNA3. A His6 tag was fused to the 3′ end, of the MSP-1₄₂ gene to allow the gene product to be affinity purified. This resulted in plasmid GTC627 (FIG. 4c).

To compare the natural MSP-1₄₂ gene construct to the modified MSP-1₄₂ nucleic acid of the invention, an expression vector was also created for the natural MSP-1₄₂ gene and the gene was added to mammalian cell culture and injected into mice to form transgenic mice as seen below.

### EXAMPLE 2

### Construction of the Native MSP-1₄₂ Expression Vector

To secrete the truncated merozoite surface protein-I (MSP-1) of Plasmodium falciparum˜the wild type gene encoding the 42 KD C-terminal part of MSP-1 (MSP-1₄₂) was fused to either the DNA sequence that encodes the first 15 or the first 26 amino acids of the goat beta-casein. This is achieved by first PCR amplify the MSP-1 plasmid (received from Dr. David Kaslow, NIH) with primers MSP1 and MSP2 (FIG. 6), then cloned the PCR product into the TA vector (Invitrogen). The BglII-XhOI fragments of the PCR product was ligated with oligos OT1 and OT2 (FIG. 6) into the expression vector pCDNA3. This yielded plasmid GTC564 (FIG. 4b), which encodes the 15 amino acid beta-casein signal peptide and the first 11 amino acids of the mature goat beta-casein followed by the native MSP-1₄₂ gene. Oligos MSP-8 and MSP-2 (FIG. 6) were used to amplify MSP-1 plasmid by PCR, the product was then cloned into TA vector. The XhoI fragment was exercised and cloned into the XhoI site of the expression vector pCDNA3 to yield plasmid GTC479 (FIG. 4a), which encoded 15 amino acid goat beta-casein signal peptide fused to the wild-type MSP-1₄₂ gene. A His6 tag was added to the 3′ end of MSP-1₄₂ gene in GTC 564 and GTC 479.

### EXAMPLE 3

### Native MSPA₄₂ Gene is not Expressed in CQS-7 Cells

Expression of the native MSP gene in cultured COS-7 cells was assayed by transient transfection assays. GTC479 and GTCS64 plasmids DNA were introduced into COS-7 cells by lipofectamine (Gibco-BRL) according to manufacturer's protocols. Total cellular RNA was isolated from the COS cells two days post-transfection. The newly synthesized proteins were metabolically labeled for 10 hours by adding ³⁵S methionine added to the culture media two days-post transfection. To determine the MSP mRNA expression in the COS cells, a Northern blot was probed with a ³²P labeled DNA fragment from GTC479. No MSP RNA was detected in GTC479 or GTC564 transfectants (data not shown). Prolonged exposure revealed residual levels of degraded MSP mRNA. The ³⁵S labeled culture supernatants and the lysates were immunoprecipitated with a polyclonal antibody raised against MSP. Immunoprecipitation experiments showed that no expression from either the lysates or the supernatants of the GTC479 or GTC564 transfected cells (data not shown). These results showed that the native MSP-1 gene was not expressed in COS cells.

### EXAMPLE 4

### Native MSP-l.s2 Gene is not Expressed in the Mammary Gland of Transgenic Mice

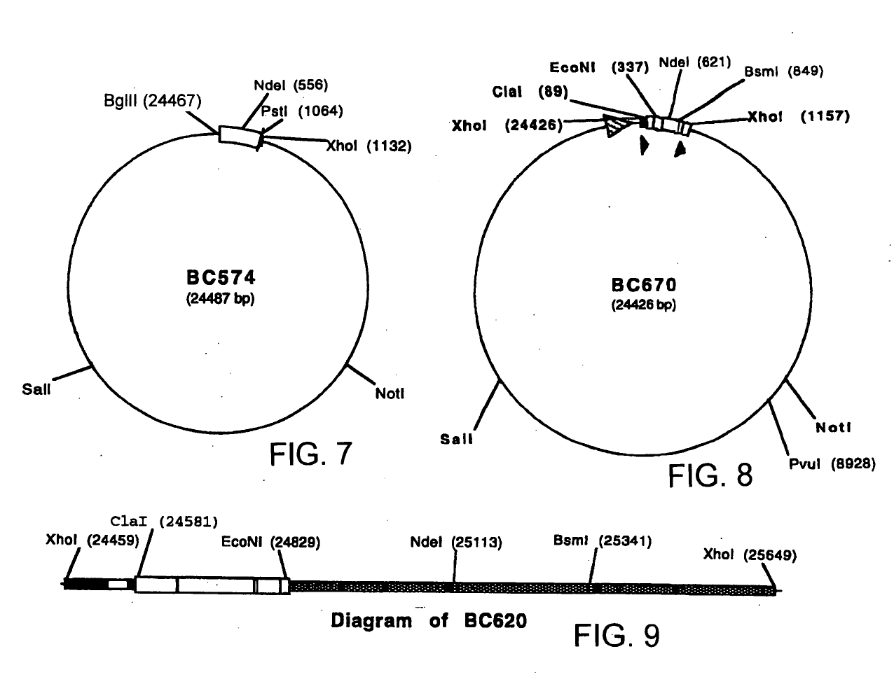

The SalI-XhoI fragment of GTC479, which encoded the 15 amino acids of goat beta-casein signal peptide, the first 11 amino acids of goat beta-casein, and the native MSP-1₄₂ gene, was cloned into the XhoI site of the beta-casein expressed in vector BC350. This yielded plasmid BC574 (FIG. 7). A SalI-NotI fragment of BC574 was injected into the mouse embryo to generate transgenic mice. Fifteen lines of transgenic mice were established. Milk from the female founder mice was collected and subjected to Western analysis with polycolonal antibodies against MSP. None of the seven mice analyzed were found to express MSP-1₄₂ protein in their milk. To further determine if the mRNA of MSP-1₄₂ was expressed in the mammary gland, total RNA was extracted from day 11 lactating transgenic mice and analyzed by Northern blotting. No MSP-1₄₂ mRNA was detected by any of the BC 574 lines analyzed. Therefore, the MSP-1₄₂ transgene was not expressed in the mammary gland of transgenic mice. Taken together, these experiments suggest that native parasitic MSP-1₄₂ gene could not be expressed in mammalian cells, and the block is as the level of mRNA abundance.

### EXAMPLE 5

### Expression of MSP in Mammalian Cells

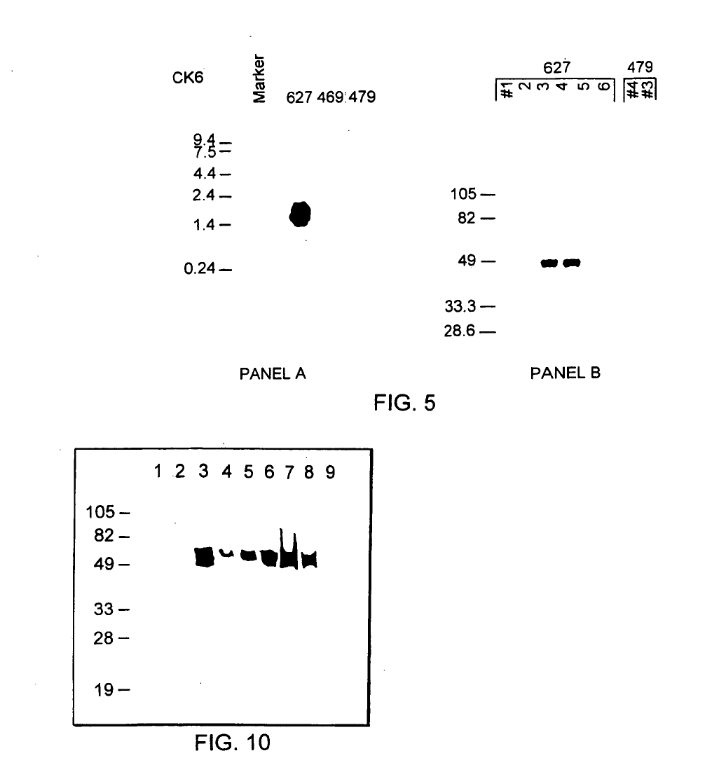

Transient transfection experiments were performed to evaluate the expression of the modified MSP-1₄₂ gene of the invention in COS cells. GTC627 and GTC479 DNA were introduced into the COS-7 cells. Total RNA was isolated 48 hours post-transfection for Northern analysis. The immobilized RNA was probed with ³²P labeled SalI-XhoI fragment of GTC627. A dramatic difference was observed between GTC479 and GTC627. While no MSP-1₄₂ mRNA was detected in the GTC479 transfected cells as shown previously, abundant MSP-1₄₂ mRNA was expressed by GTC627 (FIG. 5, Panel A). GTC 469 was used as a negative control and comprises the insert of GTC564 cloned into cloning vector PU19, a commercially available cloning vector. A metabolic labeling experiment with 355 methionine followed by immunoprecipitation with polyclonal antibody (provided by D. Kaslow NIAID, NIH) against MSP showed that MSP-1₄₂ protein was synthesized by the transfected COS cells (FIG. 5, Panel B). Furthermore, MSP-1₄₂ was detected in the transfected COS supernatant, indicating the MSP-1₄₂ protein was also secreted. Additionally, using Ni-NTA column, MSP-1₄₂ was affinity purified from the GTC627 transfected COS supernatant. These results demonstrated that the modification of the parasitic MSP-1₄₂ gene lead to the expression of MSP mRNA in the COS cells. Consequently, the MSP-1₄₂ product was synthesized and secreted by mammalian cells.

Polyclonal antibodies used in this experiment may also be prepared by means well known in the art (ANTIBODIES: A LABORATORY MANUAL, Ed Harlow and David Lane, eds. Cold Spring Harbor Laboratory, publishers (1988)). Production of MSP serum antibodies is also described in Chang et al., INFECTION AND IMMUNITY (1996) 64:253-261 and Chang et al., (1992) PROC NATL. ACAD. SCI. USA 86:6343-6347.

The results of this analysis indicate that the modified MSP-1₄₂ nucleic acid of the invention is expressed at a very high level compared to that of the natural protein which was not expressed at all. These results represent the first experimental evidence that reducing the AT % in a gene leads to expression of the MSP gene in heterologous systems and also the first evidence that removal of AUUUA mRNA instability motifs from the MSP coding region leads to the expression of MSP protein in COS cells. The results shown in FIG. 5, Panel A Northern (i.e. no RNA with native gene and reasonable levels with a modified DNA sequence in accordance with the invention), likely explains the increase in protein production. The following examples describe the expression of MSPI-42 as a native 10 non-fusion (and non-glycosylated) protein in the milk of transgenic mice.

### EXAMPLE 6

### Construction of a MSP Transgene

To fuse MSP1-42 to the 15 amino acid (3-casein signal peptide, a pair of oligos, MSP2O3 and MSP2O4 (MSP2O3: ggccgctcgacgccaccatgaaggtcctcataattgcc tgtctggtggctctggccattgcagccgtcactccctcgtcat,

MSP2O4: cgatgacggagggagtgacggctg caatggccagagccaccaga caggcaattatgaggaccttcatggtggcgtcgagc) which encode the 15 amino acid-casein signal and the first 5 amino acid of the MSP 1-42 ending at the CIa I site, was ligated with a CIa I-Xho [fragment of BC620 (FIG. 8) which encodes the rest of the MSPI-42 gene, into the Xho I site of the expression vector pCDNA3. A Xho [fragment of this plasmid (GTC669) was then cloned into the Xho I site of milk specific expression vector BC350 to generate B670 (FIG. 9)

### EXAMPLE 7

### Expression of MSP1-42 in the Milk of Transgenic Mice

A Sal I-Not I fragment was prepared from plasmid BC670 and microinjected into the mouse embryo to generate transgenic mice. Transgenic mice was identified by extracting mouse DNA from tail biopsy followed by PCR analysis using oligos GTCI7 and MSP 101 (sequences of oligos: GTC 17, GATTGACAAGTAATACGCTG1TFCCTC. Oligo MSP 101, GGATTCAATAGATACGG). Milk from the female founder transgenic mice was collected at day 7 and day 9 of lactation, and subjected to western analysis to determine the expression level of MSP-1-42 using an polyclonal anti-N4SP antibody and monoclonal anti MSP antibody 5.2 (Dr. David Kaslow. NH-I). Results indicated that the level of MSP-1-42 expression in the milk of transgenic mice was at 1-2 mg/ml (FIG. 10).

Milk is a very complex fluid composed of several phases that can be separated by centrifugation into a cream layer, an aqueous phase, and a two-phase pellet. The upper phase consists of milk cells and membranous debris, the lower of casein micelles. Casein can also be precipitated by micelle-destroying treatments such as the enzyme rennin or low pH leaving an aqueous phase that is often termed whey. If the whey is made from skim milk it is a true solution that contains all the milk sugar as well as the major milk proteins lactoferrin and secretory immunoglobulin A (sIgA), the monovalent ions sodium, potassium and chloride, citrate, calcium, free phosphate and most of the water-soluble minor components of milk. Depending on the species, varying proportions of the lactoferrin, lysozyme, citrate and calcium may be found associated with the casein pellet. The casein fraction is a small proportion of human milk, about 0.2% by weight. However, casein makes up 4% of cow's milk and as much as 12% of rodent milks. The casein fraction from cows milk, usually obtained by rennin precipitation, is used in cheese-making while the whey finds a multiplicity of uses, most notably as the base for infant formula. Taken as a feedstream, milk is an exceptionally difficult background fluid from which to purify out a single protein of interest.

### EXAMPLE 8

### Construction of MSP-1 42 Glycosylation Sites Minus Mutants

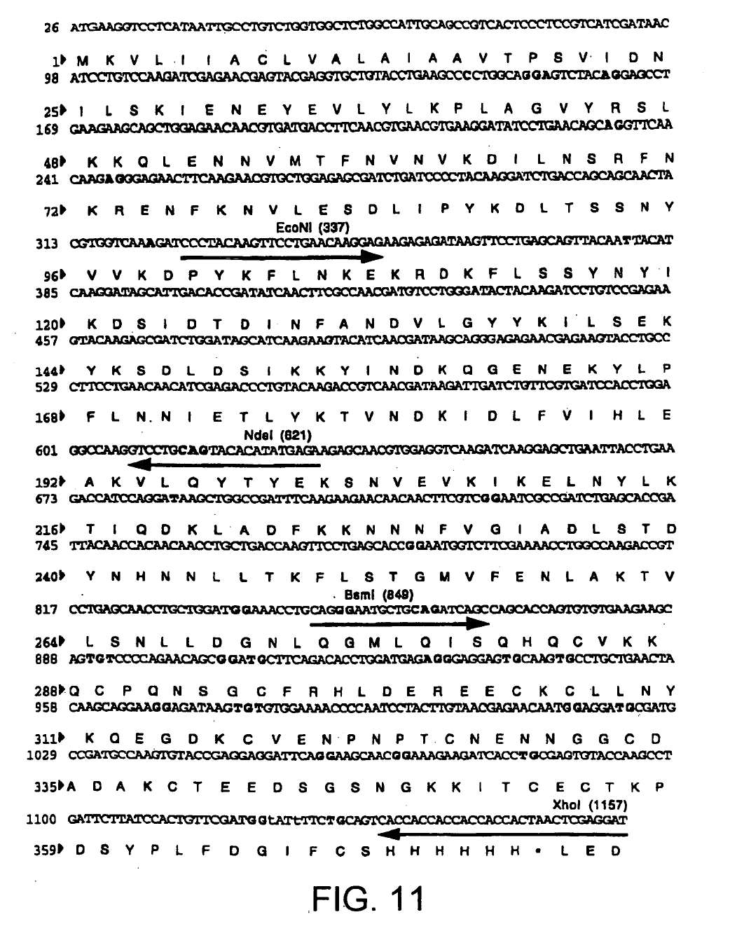

Our analysis of the milk produced MSP revealed that the transgenic MSP protein was N-glycosylated. To eliminate the N˜2lycosylation sites in the MSP 1-42 gene, Asn. (N) at positions 181 and 262 were substituted with Gln.(Q). The substitutions were introduced by designing DNA oligos that anneal to the corresponding region of MSP I and can-y the AAC to CAG mutations. These oligos were then used as PCR primers to produce DNA fragments that encode the N to Q substitutions. To introduce N262-Q mutation, a pair of oligos. MSPGYLYCO-3 (CAGGGAATGCTGCAGATCAGC) AND MSP42-2 (AAWFCTCGAGTFAGTG GTGGTGGTGGTGGTGATCGCAGAAAATACCATG. FIG. 11), were used to PCR amplify plasmid GTC627, which contains the synthetic MSPI-42 gene. The PCR product was cloned into pCR2. I vector (Invitrogen). This generated plasmidGTC716.

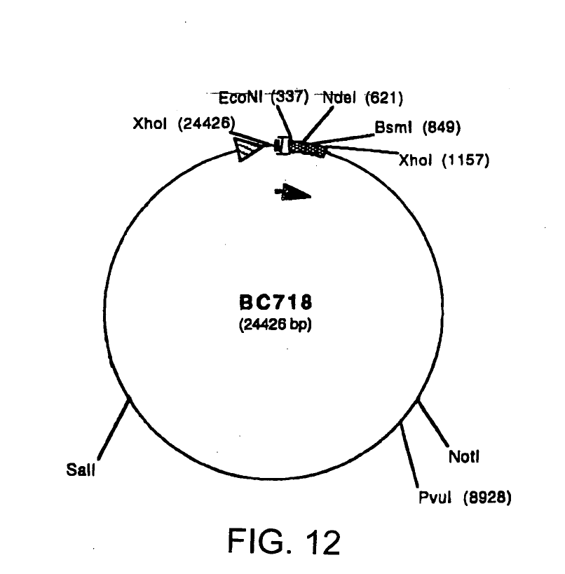

To introduce N181-Q mutation, oligos MSPGLYCO-1 (CTCCYFGTTCAGG AACTTGTAGGG) and MSPGLCO-2 (GTCCTGCAGTACACATATGAG. FIG. 4) were used to amplify plasmid GTC 627. The PCR product was cloned into pCR2. I. This generated plasmid GTC700. The MSP double glycosylation mutant was constructed by the following three steps: first, aXho I-Bsm I fragment of BC670 and the Bsm l-Xho I fragment of GTC7I6 is ligated into the Xho I site of vector pCR2.1. This resulted a plasmid that contain the MSP-1-42 gene with N262-Q mutation. EeoN I-Nde I fragment of this plasmid was then replaced by the EcoN I-Nde I fragment from plasmid GTC7I6 to introduce the second mutation. N181-Q. AXho I fragment of this plasmid was finally cloned into BC350 to generate BC7 IS (FIG. 12).

### EXAMPLE 8

### Transgenic Expression of Nonglycosylated MSP-1

BC7 18 has the following characteristics: it carries the MSP 1-42 gene under the control of the 13-casein promoter so it can he expressed in the mammary gland of the transgenic animal during lactation. Further. it encodes a 15 amino acid 13-casein leader sequence fused directly to MSP1-42 so that the N4SPI-42, without any additional amino acid at its N-terminal, can he secreted into the milk. Finally, because the N-Q substitutions, the MSP produced in the milk of the transgenic animal by this construct will not be N-glycosylated. Taken together. the transgenic N4SP produced in the milk by 8C7 18 is the same as the parasitic MSP.

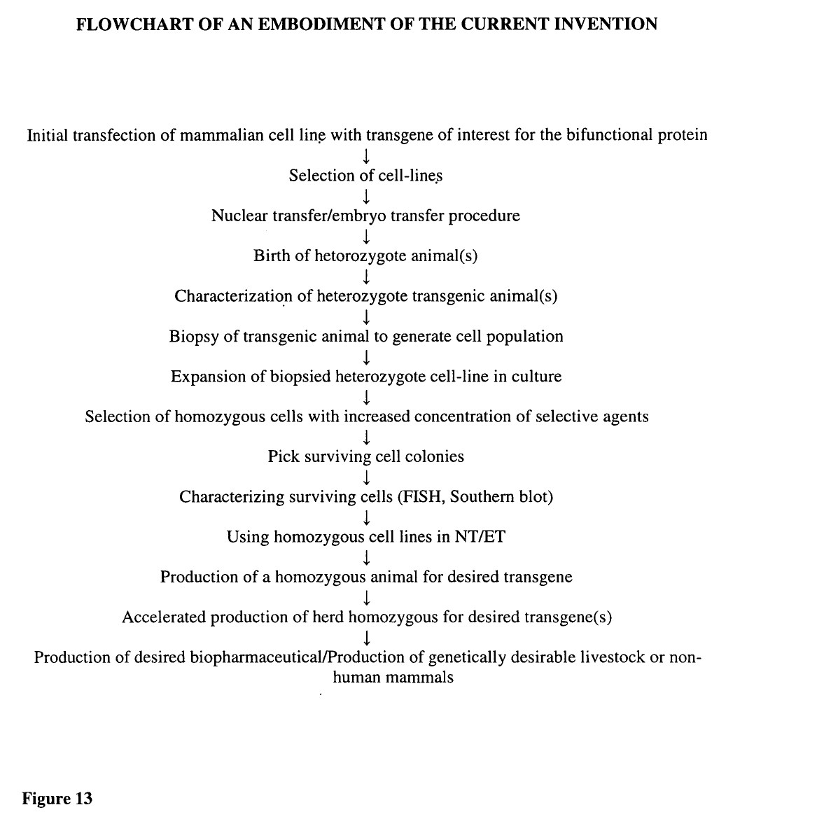

A SalL/XhoI fragment was prepared from plasmid BC7 18 and microinjected into mouse embryos to generate transgenic mice. Transgenic animals were identified as described previously. Milk from female founders was collected and analyzed by Western blotting with antibody 5.2. The results, shown in FIG. 13, indicate expression of nonglycosylated MSPI at a concentration of 0.5 to 1 mg/ml.

Malaria Protein Processing

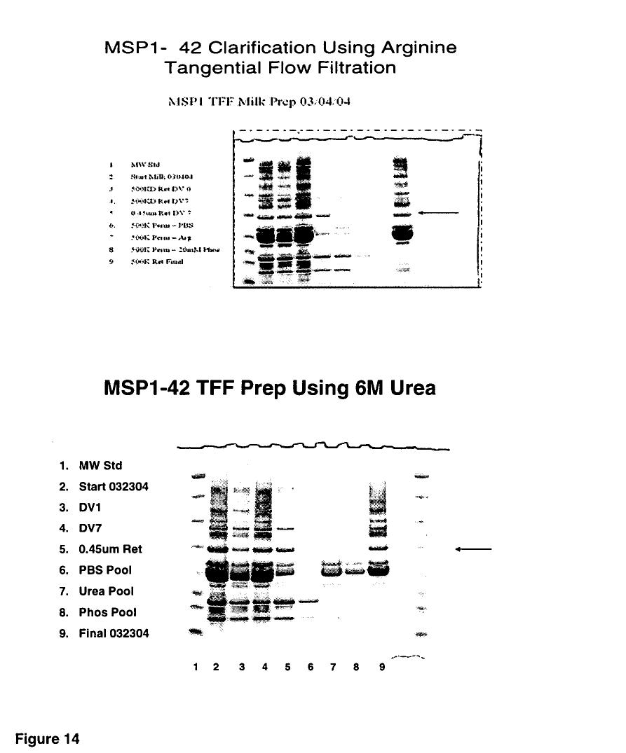

The following is a brief summary of the developments in the MSP1-42 purification process:
1. A TFF process was developed to replace the centrifuge procedure that had been used to prepare solubilized MSP1-42 from whole milk. The new process involves first adding EDTA to the whole milk and then flowing through a 0.45 um membrane. All of the proteins (including the casein-MSP1-42 micelles) flow through the 0.45 um membrane and the milk fat remains behind in the retentate.
2. The clarified filtrate is then washed on a 500 kd MWCO membrane with PBS to remove the bulk of the whey proteins (the casein-MSP 1-42 micelles remain in the retentate).
3. Arginine (0.6M) or urea (6M) is added to the washed retentate in order to solubilize the MSP1-42. This new procedure yields solubilized antigen that is superior to material from the centrifuge procedure and is quicker, easier, and more scaleable. FIG. 14 shows SDS-PAGE results of the TFF clarification process with arginine and urea.
4. The POROS HS50 column was optimized to use a simple step wash and elution. The S column is loaded in 0M NaCl pH 6.8, washed with 150 mM NaCl pH 6.8, and eluted with 150 mM NaCl pH 8.0.
5. After elution the column is regenerated with NaOH. The column appears to perform equally well with or without 0.05% Tween 80 in the buffers. Source 30S resin was compared to the POROS resin and was found to be very inferior. Several HIC resins were tested as a second step in the purification process. Even when the columns were loaded at very high salt concentrations the MSP1-42 was difficult to bind. In any event, and according to the methods of the prior art, the recovery of MSP1-42 and resolution from contaminants was often poor. These difficulties were overcome by the methods of the current invention.

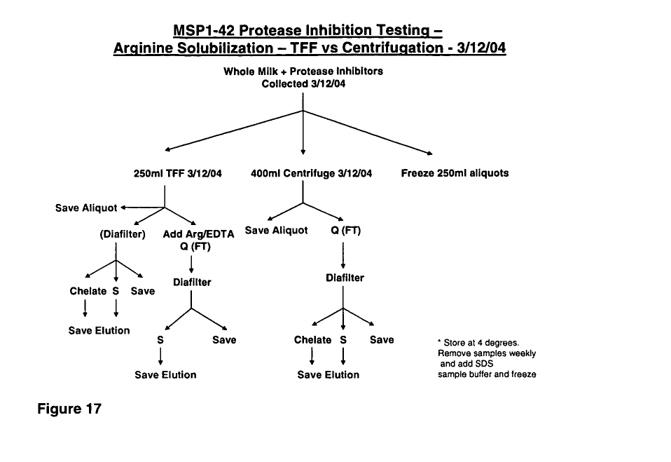

Turning to FIG. 16, from this experiment we can see that it takes at least 2-3 weeks before the first evidence of protein degradation can be seen. This should not be a problem in real life since we will not let solubilized antigen preps sit around this long without further processing. Degradation studies have been done with and without protease inhibitors, comparing centrifugation and TFF with arginine solubilization, and comparing TFF with arginine and urea solubilization. Solubilized samples were stored at 4° C. as is and purified through various chromatography steps. FIGS. 17, 18 and 19 show outlines of the degradation studies. The results showed that the addition of protease inhibitors significantly reduced the amount of degradation in all samples. Using TFF instead of centrifugation also reduced the amount of degradation even without protease inhibitors. The TFF samples that had been solubilized with urea were the best of all.

According to the current invention the purification process developed with a non-his tagged MSP1-42 from E coli involved solubilizing the E coli inclusion bodies with urea and then maintaining a low level of urea in the column buffers of at least the first few steps. This method has the benefit of both maintaining the solubility of the antigen as well as potentially inhibiting protease activity. Process development for the preferred methods of the current invention involve using urea.

The TFF procedure for solubilizing MSP1-42 has been modified to use 6M urea. As we have shown in the past, the antigen can be solubilized with high concentrations of urea just as well as with arginine. In a preferred embodiment of the current invention 6M urea is substituted for 0.6M arginine after the wash step. The final solubilized antigen is then diafiltered into phosphate buffer containing 1M urea as well as 0.05% tween 80. Recovery and quality with the urea process is superior to the arginine process.

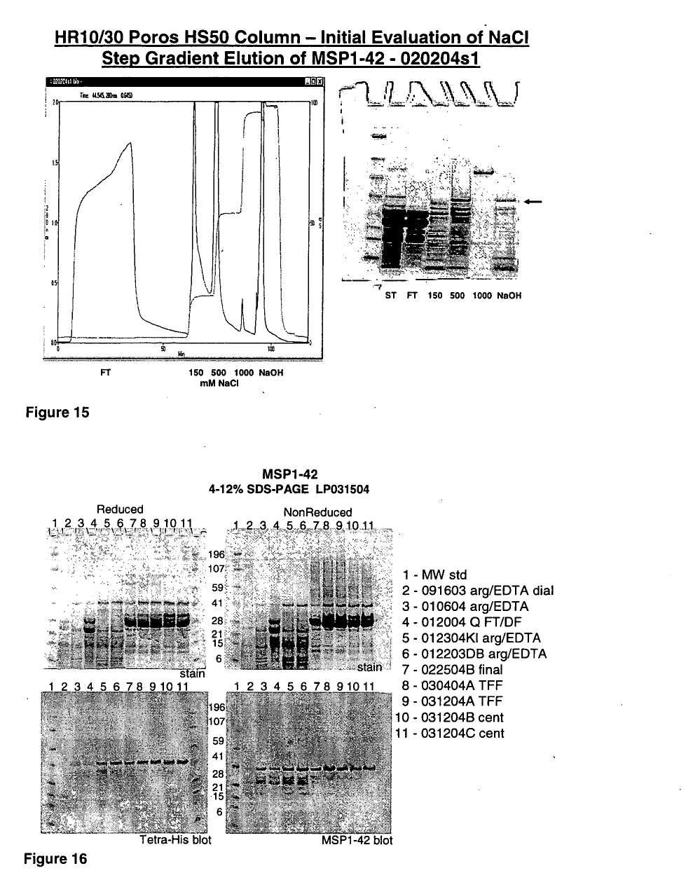

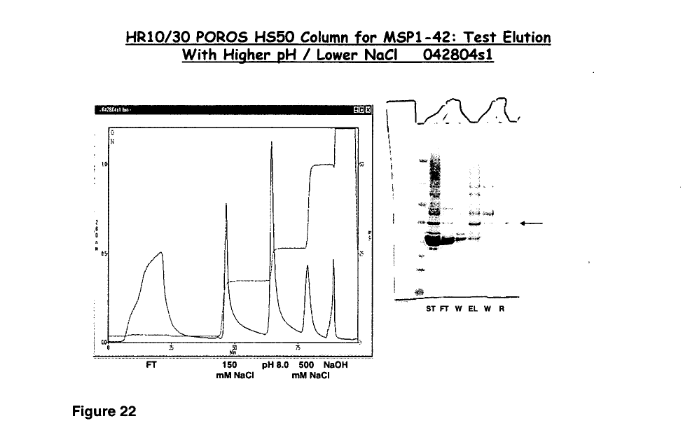

The POROS HS50 column continues to be the first chromatography step in the purification process. The basic procedure remains the same except 0.5M urea has been added to the equilibration and elution buffers. The presence of urea has no impact on the performance of the column. FIG. 15 shows the results of an initial POROS HS50 column using arginine-solubilized MSP1-42 and elution with 500 mM NaCl at pH 6.8. This column removes a great deal of casein as well as other minor contaminants. FIG. 22 shows the results of a reoptimized POROS HS50 column with urea and using pH 8.0 for elution. The use of urea and modification of the elution conditions has improved the purification value of this column.

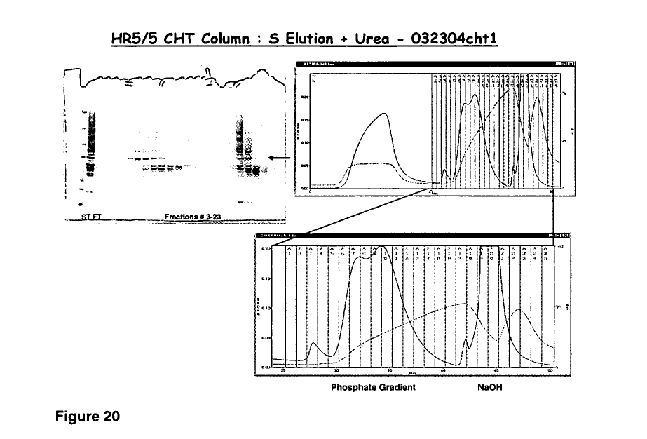

Hydroxyapatite (CHT) has been tested as a second step in the purification process. The S column elution fraction can be diluted and loaded directly onto the CHT column. Initial experiments looked at using a 20-500 mM phosphate gradient elution similar to the CHT column used in the old process but with 0.5M urea in the buffers. The results of the first run can be seen in FIG. 20. The results, according to the current invention, provide a substantial improvement for the purification process of the invention.

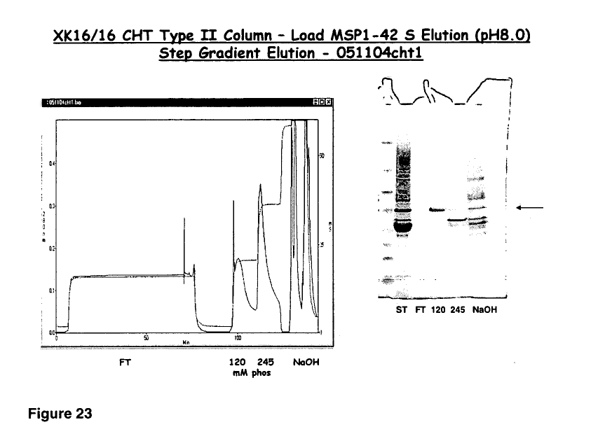

The CHT column was optimized and elution was converted from a linear gradient to a step. FIG. 23 shows the final result: a column with good purification value. At loads of up to 7.5 mg total protein per ml resin, no MSP1-42 is seen in the flow through.

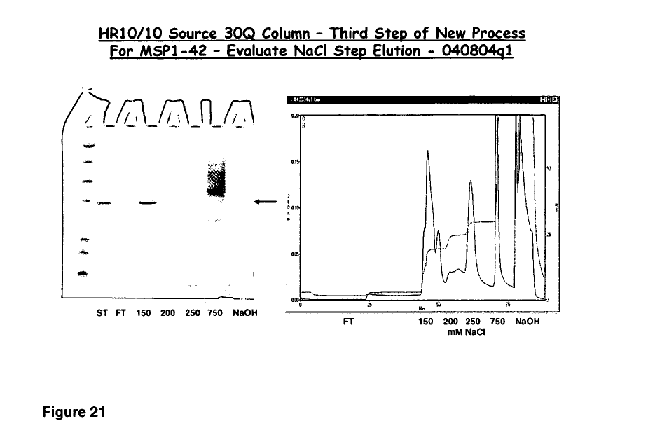

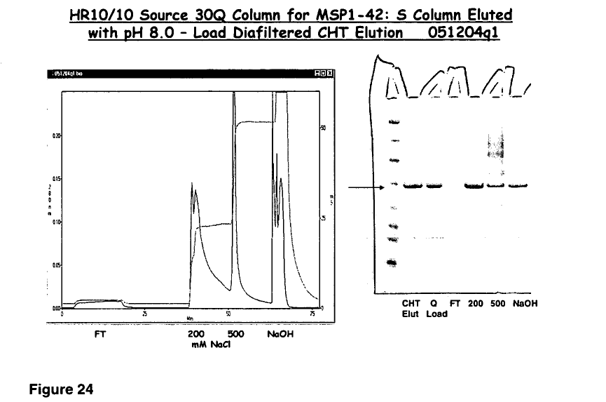

The Q column is the logical choice for the third step in the new process since it performed so well as the third step of the old process. FIG. 21 shows the results of an early Source 30Q column using a NaCl step elution, and FIG. 24 shows the results of a reoptimized Source 30Q column with urea in the column buffers. The elution fraction from the CHT column will need to be diluted or diafiltered before loading onto the Q column.

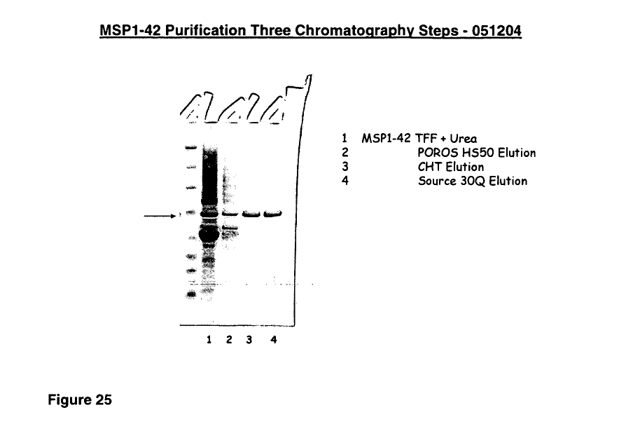

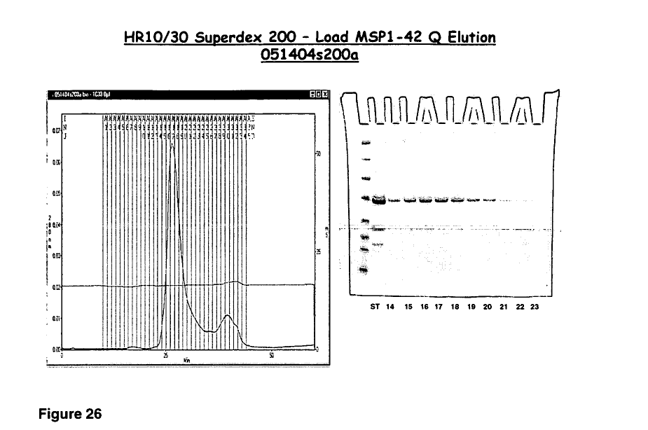

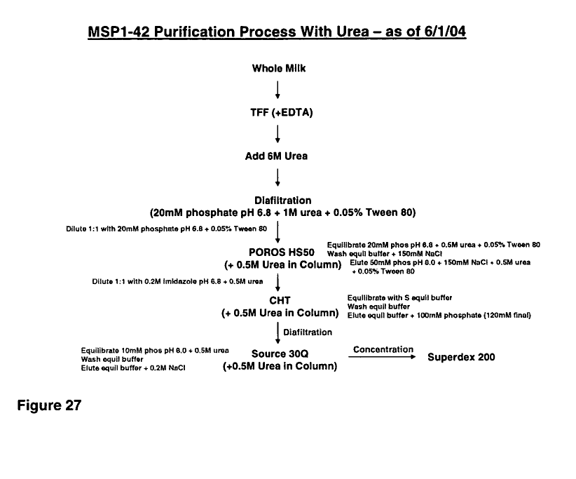

FIG. 27 shows an outline of the new purification process as it appears to be shaping up and FIG. 25 shows SDS-PAGE of MSP1-42 through the first three steps of the purification process. The Superdex 200 column has been evaluated as a final polishing step and does appear to add to the quality of the final product. FIG. 26 shows the results of a Superdex 200 column. The elution fraction from the Q column will need to be concentrated prior to loading onto the Superdex 200 column.

Methods & Processes

Prion Safety

Transmissible spongiform encephalopathies (TSE), such as nvCJD in humans, BSE in cattle and scrapie in sheep and goats, also must be considered in assuring the safety of products made from human or ruminant sources. Human donors are monitored for CJD and nvCJD and potentially contaminated blood, plasma pools and products made from them have been recalled or traced when a contributing donor has been diagnosed with CJD. All transgenic goats used as the source of a biopharmaceutical by the inventors are certified free of scrapie in the 5 yr USDA Voluntary Scrapie Certification Program and various risk minimization measures have been instituted to reduce any potential risk from this TSE in this highly controlled donor closed donor goat population. In addition, the MSP-1 purification process has been validated for its ability to remove ≧11.3 log₁₀ scrapie.

Transgenic Production of MSP-1

Expression of Construct

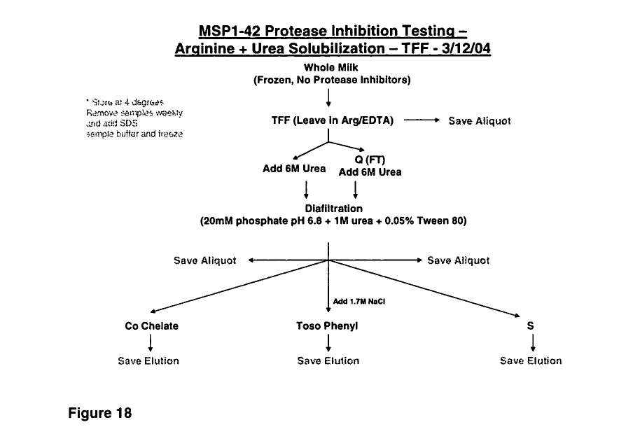

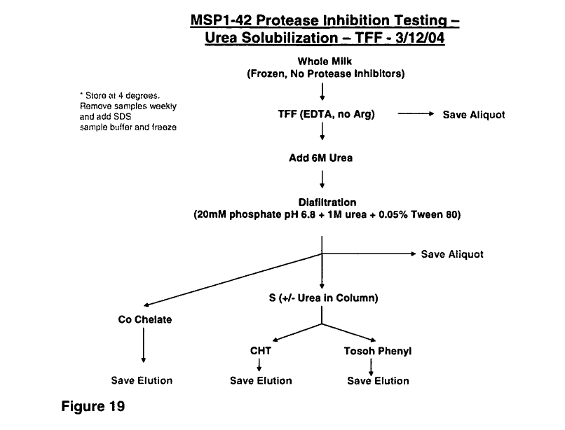

The MSP-1 transgenic goats contain the cDNA for MSP-1 altered for production in a mammalian system (derived from a modified pBAT6 plasmid-Broker et al 1987) isolated from a human cDNA library in a goat beta casein expression cassette (FIG. 18). This milk specific expression cassette is routinely used by GTC for production of transgenic animals that express human therapeutic proteins in their milk. This expression construct was microinjected into pronuclei of fertilized goat eggs (FIG. 19), which were then transferred to the oviducts of pseudopregnant recipients. The gestation period in the goat is 150 days. Three generations of his female offspring have produced the milk used for purification of MSP-1.

The Purification Process

The source material for purification is milk produced by transgenic goats expressing the MSP-1 protein at approximately 2 g/L. Goats typically lactate 300 days per year producing 2-4 liters of milk per day. The process normally produces 300 grams of purified MSP-1 per batch from no more than 375 liters of milk containing approximately 600 grams of MSP-1. The volume of milk processed per batch is determined based on the MSP-1 binding capacity of the initial Heparin (Heparin-Hyper D) column and the MSP-1 concentration of the milk. The full MSP-1 purification process is depicted in FIG. 18.

Upstream processing is defined as thawing, pooling and clarification of the source material (milk) and initial mass capture. Milk containing MSP-1 is diluted with an equal weight of EDTA buffer and is then clarified by tangential flow filtration with a nominal 500-kDa-pore size Hollow Fiber membrane filter (VirA/Gard 500-kDa) (Step 1). The VirA/Gard 500-kDa permeate is cycled through a closed loop linking the filtration system to the Heparin-Hyper D column (Step 2) until >90% of the MSP-1 is captured (about 8 volume cycles). (Note! Bayer uses the same Heparin Hyper D column in their process for Thrombate (Lebing et al 1994).

The Heparin-Hyper D column is washed and then eluted with a 2.5 M sodium chloride buffer. Once the Heparin-Hyper D eluate is obtained, it is transferred into a downstream processing and formulation area. Recently a Pall DV-20 viral removal filter (Step 3) has been validated and inserted into the process following the heparin column and prior to the ANX ion exchange column

Downstream processing (FIG. 20) includes concentration and diafiltration of the Heparin-Hyper D column eluate, ion exchange chromatography (ANX-Sepharose), hydrophobic interaction chromatography (Methyl HyperD) and a second concentration and diafiltration step. After the heparin eluate is transferred into the downstream processing area, it is filtered through a Pall DV-20 viral removal filter, concentrated and diafiltered by membrane filtration to adjust the ionic strength for the application of the MSP-1 onto the ANX-Sepharose column. After loading, the ANX-Sepharose column is washed and the MSP-1 is eluted with 0.32 M buffer. The ANX-Sepharose eluate is conditioned with sodium citrate and applied to the Methyl HyperD column. The Methyl HyperD column is washed and the MSP-1 is eluted from the resin with a lower concentration sodium citrate buffer.

Final formulation is achieved by concentration and diafiltration into a citrate, glycine, sodium chloride buffer with the proper ionic strength and dilution to the final protein concentration of approximately 25 mg/ml. This formulation is the same one used by Aventis Behring for Kybernin P. Formulated batches are tested, and those meeting the product specifications are pooled to meet the lot size requirements. The product is filled into vials (10 ml containing approximately 250 mg protein), lyophilized and then heat treated in a validated controlled temperature oven at 80° C. for 72 hours in a validated terminal viral inactivation step.

Transgenic Goats & Cattle

The herds of pure- and mixed-breed scrapie-free Alpine, Saanen and Toggenburg dairy goats used as cell and cell line donors for this study were maintained under Good Agricultural Practice (GAP) guidelines. Similarly, cattle used should be maintained under Good Agricultural Practice (GAP) guidelines and be certified to originate from a scrapie and bovine encephalitis free herd.

Isolation of Caprine Fetal Somatic Cell Lines.

Primary caprine fetal fibroblast cell lines to be used as karyoplast donors were derived from 35- and 40-day fetuses. Fetuses were surgically removed and placed in equilibrated phosphate-buffered saline (PBS, Ca⁺⁺/Mg⁺⁺-free). Single cell suspensions were prepared by mincing fetal tissue exposed to 0.025 % trypsin, 0.5 MM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I.U. each/ml)], and were cultured in 25 cm² flasks. A confluent monolayer of primary fetal cells was harvested by trypsinization after 4 days of incubation and then maintained in culture or cryopreserved.

Preparation of Donor Cells for Embryo Reconstruction.

Transfected fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 2-7 days following low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) for at least 6 hours prior to transgenic to the enucleated oocytes. The current experiments for the generation of desirable transgenic animals are preferably carried out with goat cells or mouse cells for the generation or goats or mice respectively but, according to the current invention, could be carried out with any mammalian cell line desired.

Oocyte Collection.

Oocyte donor does were synchronized and super ovulated as previously described (Ongeri, et al., 2001), and were mated to vasectomized males over a 48-hour interval. After collection, oocytes were cultured in equilibrated M199 with 10% FBS supplemented with 2 mM L-glutamine and 1% penicillin/streptomycin (10,000 l.U. each/ml).

Cytoplast Preparation and Enucleation.

All oocytes were treated with cytochalasin-B (Sigma, 5 μg/ml in SOF with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in SOF for 30 to 60 minutes before electrotransgenic and activation procedures. Reconstructed oocytes were equilibrated in transgenic buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrotransgenic and activation were conducted at room temperature, in a transgenic chamber with 2 stainless steel electrodes fashioned into a “transgenic slide” (500 μm gap; BTX-Genetronics, San Diego, Calif.) filled with transgenic medium.

Transgenic was performed using a transgenic slide. The transgenic slide was placed inside a transgenic dish, and the dish was flooded with a sufficient amount of transgenic buffer to cover the electrodes of the transgenic slide. Couplets were removed from the culture incubator and washed through transgenic buffer. Using a stereomicroscope, couplets were placed equidistant between the electrodes, with the karyoplast/cytoplast junction parallel to the electrodes. It should be noted that the voltage range applied to the couplets to promote activation and transgenic can be from 1.0 kV/cm to 10.0 kV/cm. Preferably however, the initial single simultaneous transgenic and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh transgenic buffer. Transgenic treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Nuclear Transfer Embryo Culture and Transfer to Recipients

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Meng et al., 1997), have also been reported. The stage of cell cycle that the karyoplast is in at time of reconstruction has also been documented as critical in different laboratories methodologies (Kasinathan et al., BIOL. REPROD. 2001; Yong et al., 1998; and Kasinathan et al., NATURE BIOTECH. 2001).

All nuclear transfer embryos of the current invention were cultured in 50 μl droplets of SOF with 10% FBS overlaid with mineral oil. Embryo cultures were maintained in a humidified 39° C. incubator with 5% CO₂ for 48 hours before transfer of the embryos to recipient does. Recipient embryo transfer was performed as previously described (Baguisi et al., 1999).

Paramount to the success of any nuclear transfer program is having adequate transgenic of the karyoplast with the enucleated cytoplast. Equally important however is for that reconstructed embryo (karyoplast and cytoplast) to behave as a normal embryo and cleave and develop into a viable fetus and ultimately a live offspring. Results from this lab detailed above show that both transgenic and cleavage either separately or in combination have the ability to predict in a statistically significant fashion which cell lines are favorable to nuclear transfer procedures. While alone each parameter can aid in pre-selecting which cell line to utilize, in combination the outcome for selection of a cell line is strengthened.

Pregnancy and Perinatal Care.

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF2μ (Lutalyse, Upjohn). Parturition occurred within 24 hours after treatment. Kids were removed from the dam immediately after birth, and received heat-treated colostrum within 1 hour after delivery. Time frames appropriate for other ungulates with regard to pregnancy and perinatal care (e.g., bovines) are known in the art.

Animal Promoters

Useful promoters for the expression of a target protein the mammary tissue include promoters that naturally drive the expression of mammary-specific polypeptides, such as milk proteins. These include, e.g., promoters that naturally direct expression of whey acidic protein (WAP), alpha S1-casein, alpha S2-casein, beta-casein, kappa-casein, beta-lactoglobulin, alpha-lactalbumin (see, e.g., Drohan et al., U.S. Pat. No. 5,589,604; Meade et al., U.S. Pat. No. 4,873,316; and Karatzas et al., U.S. Pat. No. 5,780,009), and others described in U.S. Pat. No. 5,750,172. Whey acidic protein (WAP; Genbank Accession No. XO1153), the major whey protein in rodents, is expressed at high levels exclusively in the mammary gland during late pregnancy and lactation (Hobbs et al., J. BIOL. CHEM. 257:3598-3605, 1982). For additional information on desired mammary gland-specific promoters, see, e.g., Richards et al., J. BIOL. CHEM. 256:526-532, 1981 (α-lactalbumin rat); Campbell et al., NUCLEIC ACIDS RES. 12:8685-8697, 1984 (rat WAP); Jones et al., J. BIOL. CHEM. 260:7042-7050, 1985 (rat β-casein); Yu-Lee & Rosen, J. BIOL. CHEM. 258:10794-10804, 1983 (rat γ-casein); Hall, BIOCHEM. J. 242:735-742, 1987 (human α-lactalbumin); Stewart, NUCLEIC ACIDS RES. 12:3895-3907, 1984 (bovine α-s1 and κ-casein cDNAs); Gorodetsky et al., GENE 66:87-96, 1988 (bovine β-casein); Alexander et al., EUR. J. BIOCHEM. 178:395-401, 1988 (bovine κ-casein); Brignon et al., FEBS LETT. 188:48-55, 1977 (bovine α-S2 casein); Jamieson et al., GENE 61:85-90, 1987, Ivanov et al., BIOL. CHEM. Hoppe-Seyler 369:425-429, 1988, and Alexander et al., NUCLEIC ACIDS RES. 17:6739, 1989 (bovine β-lactoglobulin); and Vilotte et al., BIOCHIMIE 69:609-620, 1987 (bovine α-lactalbumin). The structure and function of the various milk protein genes are reviewed by Mercier & Vilotte, J. DAIRY SCI. 76:3079-3098, 1993.

Other promoters that are useful in the methods of the invention include inducible promoters. Generally, recombinant proteins are expressed in a constitutive manner in most eukaryotic expression systems. The addition of inducible promoters or enhancer elements provides temporal or spatial control over expression of the transgenic proteins of interest, and provides an alternative mechanism of expression. Inducible promoters include heat shock protein, metallothionien, and MMTV-LTR, while inducible enhancer elements include those for ecdysone, muristerone A, and tetracycline/doxycycline.

The proteins of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or excipients. Thus, the malaria related molecules and their physiologically acceptable salts and solvate may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they maybe presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner.

For administration by inhalation, the malaria related molecules for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The malaria related transgenic proteins of the invention may be formulated for parenteral administration by injection, e.g., by bolus injection or continuous intransgenic. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In addition to the formulations described previously, the malaria related molecules may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Treatment Methods

Therapeutic methods involve administering to a subject in need of treatment a therapeutically effective amount of a transgenic protein. “Therapeutically effective” is employed here to denote the amount of transgenic proteins that are of sufficient quantity to inhibit or reverse a disease condition (e.g., reduce or inhibit cancer growth or acquired deficiency). Administration during in vivo treatment may be by any number of routes, including parenteral and oral, but preferably parenteral. Intracapsular, intravenous, intrathecal, and intraperitoneal routes of administration may be employed, generally intravenous is preferred. The skilled artisan will recognize that the route of administration will vary depending on the disorder to be treated.

Determining a therapeutically effective amount specifically will depend on such factors as toxicity and efficacy of the medicament. Toxicity may be determined using methods well known in the art and found in the foregoing references. Efficacy may be determined utilizing the same guidance in conjunction with the methods described below in the Examples. A pharmaceutically effective amount, therefore, is an amount that is deemed by the clinician to be toxicologically tolerable, yet efficacious. Efficacy, for example, can be measured by the induction or substantial induction of T lymphocyte cytotoxicity at the targeted tissue or a decrease in mass of the targeted tissue. Suitable dosages can be from about 1 mg/kg to 10 mg/kg.

The foregoing is not intended to have identified all of the aspects or embodiments of the invention nor in any way to limit the invention. The accompanying drawings, which are incorporated and constitute part of the specification, illustrate embodiments of the invention, and together with the description, serve to explain the principles of the invention.

All publications and patent applications mentioned in this specification are herein incorporated by reference to the same extent as if each independent publication or patent application is specifically indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

Those skilled in the art will be able to recognize, or be able to ascertain using no more than routine experimentation, numerous equivalents to the specific procedures described in the embodiments of the invention provided herein. Such equivalents are considered to be within the scope of this invention and are covered by the appended claims.

Moreover, although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

### LITERATURE CITED AND INCORPORATED BY REFERENCE

1. Baguisi A, (1999) et al., Production of Goats by Somatic Cell Nuclear Transfer, NATURE BIOTECH; 17: 456-461.

2. Groner, A., Schmitt, K., and Schuler, E. (1999) Elimination of parvovirus B19 by the manufacturing process of Kybernin P. Ann. Hematol. 78 (Suppl. 1):A90.

3. Harper, P. L., Evans, D., and Carrell, R. W. (1997) The potential hazards of MSP-1 preparations.

4. REMINGTON'S PHARMACEUTICAL SCIENCES (16^{th} ed., Osol, A., editor; Mack, Easton Press. (1980)).

5. Sambrook et al., MOLECULAR CLONING—A LABORATORY MANUAL, Cold Spring Harbor Laboratory Press, (2nd Edition) (1989).

6. Wilmut I, et al., (2002) Somatic Cell Nuclear Transfer, NATURE 10;419(6907):583-6.

7. Wilmut I, et al., (1997) Viable Offspring Derived From Fetal and Adult Mammalian Cells, NATURE February 27;385(6619):810-3.

8. Zou X, et al.,(2002) Generation of Cloned Goats (Capra Hircus) from Transfected Foetal Fibroblast Cells, The Effect of Donor Cell Cycle, MOL REPROD DEV.; 61: 164-172.

9. Prado S M, et al., (1999), Development And Validation Study For The Chromatographic Purification Process For Tetanus Anatoxin On Sephacryl S-200 High Resolution, BOLL CHIM FARM. 138(7):364-368.

10. Strauss P R (1995), Use Of Filtron Mini-Ultrasettetm Tangential Flow Device And Filtron Microseptm Centrifugal Concentrators In The Early Stages Of Purification Of DNA Polymerases, BIOTECHNIQUES 18(1):158-160.

11. Gabler et al., (1987), Principles of Tangential Flow Filtration: Applications to Biological Processing, in FILTRATION IN THE PHARMACEUTICAL INDUSTRY, pp. 453-490.

### PATENTS CITED AND INCORPORATED BY REFERENCE

1. Lenk, et al., U.S. Pat. No. 5,948,441.

2. Marinaccio, et al., U.S. Pat. No. 4,888,115.

3. Meade, et al., U.S. Pat. No. 4,873,316.

4. Meade, et al., U.S. Pat. No. 5,750,172.

5. Meade, et al., U.S. Pat. No. 5,827,690.

6. Stice, et al., U.S. Pat. No. 5,945,577

7. van Reis, et al., U.S. Pat. No. 5,256,294.

8. van Reis, et al., U.S. Pat. No. 5,490,937.

What is claimed is:

1. A method of purifying a merozite surface protein 1 (MSP-1) or a fragment thereof from a feedstream, comprising:
Solubilizing said merozite surface protein 1 from a feedstream utililizing a tangential flow filtration process;
Washing said filtrate on a membrane with a PBS solution wherein said MSP-1 or fragment thereof remains in the retentate on the purification column;
Adding an aqueous arginine or urea solution to said MSP-1 remaining in the retentate to solubilize it;
Eluting said MSP-1 from said purification column; and,
Purifying out said MSP-1 from elution

2. The method of claim 1 further comprising regenerating a purification column after the addition of said arginine or urea solution by using an aqueous NaOH solution.

3. The method of claim 1 wherein said tangential flow filtration process also includes the use of an EDTA solution.

4. The method of claim 1 wherein the MSP-1 finally eluted from the column is thereafter stored at 4° C.

5. The method of claim 1 wherein the resin used is selected from the list comprising POROS HS-50 resin; CHT resin; and, 30Q resin.

6. The method of claim 1 wherein said purification protocol has Tween 80 in the buffers.

7. The method of claim 1 wherein said purification protocol has no Tween in the buffers.

8. The method of claim 1 wherein said purification protocol has 0.05% Tween 80 in the buffers.

9. The method of claim 1 further comprising wherein a Q column is used as an initial flow through step prior to said Tangential Flow Filtration process.

10. The method of claim 1 wherein said arginine solution is between 0.2 and 1.0 M in concentration.

11. The method of claim 10 wherein said arginine solution is 0.6M in concentration.

12. The method of claim 1 wherein the clarified filtrate, after capture is then washed on a 500 kd MWCO membrane with PBS to remove the bulk of remaining proteins.

13. The method of claim 1 wherein said feedstream is milk.

14. The method of claim 1 wherein said tangential flow filtration process also includes the use of a membrane of sufficient pore size to retain milk fat while allowing the flow through of casein proteins.

15. The method of claim 1 wherein said membrane is at least 0.45 um in pore size.

16. The method of claim 1 wherein said merozite surface protein 1 (MSP-1) or a fragment thereof is produced in the milk of a non-human transgenic mammal

17. The method of claim 1 wherein said non-human transgenic mammal possesses a genome further comprising a modified nucleic acid encoding MSP-1 or fragment thereof operably linked to a promoter which directs expression in the mammary gland, wherein the nucleic acid has been modified by replacing at least one AT-containing codon of a wild-type nucleic acid sequence encoding MSP-1 with a preferred codon encoding the same amino acid as the replaced codon such that the AT-content of the modified nucleic acid is lowered as compared to the wild-type nucleic acid sequence encoding MSP-1.

18. The method of claim 1 wherein the material removed in the partial purification of step (b) is selected from the group consisting of lipids, small molecule contaminants, proteins other than MSP-1, bacterial contaminants, viral contaminants and any combination thereof.

19. The method of claim 18 wherein the material removed in the partial purification of step (b) is one or more of the materials selected from the group consisting of cream, casein, lipids, small molecule contaminants, and proteins other than MSP-1.


SEQUENCE LISTING (8 sequences)

SEQ ID NO: 1 - DNA, 1065 bp, Unknown
  Oligonucleotide
gccgtcactccctccgtcatcgataacatcctgtccaagatcgagaacgagtacgaggtgctgtacctgaagccgctggcaggggtctaccggagcctgaagaagcagctggagaacaacgtgatgaccttcaacgtgaacgtgaaggatatcctgaacagccggttcaacaagcgggagaacttcaagaacgtgctggagagcgatctgatcccctacaaggatctgaccagcagcaactacgtggtcaaggatccctacaagttcctgaacaaggagaagagagataagttcctgagcagttacaactacatcaaggatagcattgataccgatatcaacttcgccaacgatgtcctgggatactacaagatcctgtccgagaagtacaagagcgatctggattcaatcaagaagtacatcaacgataagcagggagagaacgagaagtacctgcccttcctgaacaacatcgagaccctgtacaagaccgtcaacgataagattgatctgttcgtgatccacctggaggccaaggtcctgaactacacatatgagaagagcaacgtggaggtcaagatcaaggagctgaattacctgaagaccatccaggataagctggccgatttcaagaagaacaacaacttcgtcgggatcgccgatctgagcaccgattacaaccacaacaacctgctgaccaagttcctgagcaccggtatggtcttcgaaaacctggccaagaccgtcctgagcaacctgctggatgggaacctgcaggggatgctgaacatcagccagcaccagtgtgtgaagaagcagtgtccccagaacagcgggtgtttcagacacctggatgagagagaggagtgtaagtgtctgctgaactacaagcaggaaggtgataagtgtgtggaaaaccccaatcctacttgtaacgagaacaatggtggatgtgatgccgatgccaagtgtaccgaggaggattcagggagcaacgggaagaagatcacctgtgagtgtaccaagcctgattcttatccactgttcgatggtatcttctgtagt

SEQ ID NO: 2 - DNA, 1086 bp, Unknown
  oligonucleotide
gcagtaactccttccgtaattgataacatactttctaaaattgaaaatgaatatgaggttttatatttaaaacctttagcaggtgtttatagaagtttaaaaaaacaattagaaaataacgttatgacatttaatgttaatgttaaggatattttaaattcacgatttaataaacgtgaaaatttcaaaaatgttttagaatcagatttaattccatataaagatttaacatcaagtaattatgttgtcaaagatccatataaatttcttaataaagaaaaaagagataaattcttaagcagttataattatattaaggattcaatagatacggatataaattttgcaaatgatgttcttggatattataaaatattatccgaaaaatataaatcagatttagattcaattaaaaaatatatcaacgacaaacaaggtgaaaatgagaaataccttccctttttaaacaatattgagaccttatataaaacagttaatgataaaattgatttatttgtaattcatttagaagcaaaagttctaaattatacatatgagaaatcaaacgtagaagttaaaataaaagaacttaattacttaaaaacaattcaagacaaattggcagattttaaaaaaaataacaatttcgttggaattgctgatttatcaacagattataaccataataacttattgacaaagttccttagtacaggtatggtttttgaaaatcttgctaaaaccgttttatctaatttacttgatggaaacttgcaaggtatgttaaacatttcacaacaccaatgcgtaaaaaaacaatgtccacaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccaaatgtaccgaagaagattcaggtagcaacggaaagaaaatcacatgtgaatgtactaaacctgattcttatccacttttcgatggtattttctgcagtcaccaccaccaccaccactaa

SEQ ID NO: 3 - DNA, 85 bp, Unknown
  oligonucleotide
tcgacgagagccatgaaggtcctcatccttgcctgtctggtggctctggccattgcaagagagcaggaactcaatgtcgtcggta

SEQ ID NO: 4 - DNA, 88 bp, Unknown
  oligonucleotide
gatctaccgactacattgagttcttcctgctctcttgcaatggccagagccaccagacaggcaaggatgaggaccttcatggctctcg

SEQ ID NO: 5 - DNA, 60 bp, Unknown
  oligonucleotide
taactcgagcgaaccatgaaggtcctcatccttgcctgtctggtggctctggccattgca

SEQ ID NO: 6 - DNA, 48 bp, Unknown
  oligonucleotide
aattctcgagttagtggtggtggtggtggtgactgcagaaataccatc

SEQ ID NO: 7 - DNA, 31 bp, Unknown
  oligonucleotide
aatagatctgcagtaactccttccgtaattg

SEQ ID NO: 8 - DNA, 1136 bp, Unknown
  oligonucleotide
atgaaggtcctcataattgcctgtctggtggctctggccattgcagccgtcactccctccgtcatcgataacatcctgtccaagatcgagaacgagtacgaggtgctgtacctgaagcccctggcaggagtctacaggagcctgaagaagcagctggagaacaacgtgatgaccttcaacgtgaacgtgaaggatatcctgaacagcaggttcaacaagagggagaacttcaagaacgtgctggagagcgatctgatcccctacaaggatctgaccagcagcaactacgtggtcaaagatccctacaagttcctgaacaaggagaagagagataagttcctgagcagttacaattacatcaaggatagcattgacaccgatatcaacttcgccaacgatgtcctgggatactacaagatcctgtccgagaagtacaagagcgatctggatagcatcaagaagtacatcaacgataagcagggagagaacgagaagtacctgcccttcctgaacaacatcgagaccctgtacaagaccgtcaacgataagattgatctgttcgtgatccacctggaggccaaggtcctgcagtacacatatgagaagagcaacgtggaggtcaagatcaaggagctgaattacctgaagaccatccaggataagctggccgatttcaagaagaacaacaacttcgtcggaatcgccgatctgagcaccgattacaaccacaacaacctgctgaccaagttcctgagcaccggaatggtcttcgaaaacctggccaagaccgtcctgagcaacctgctggatggaaacctgcagggaatgctgcagatcagccagcaccagtgtgtgaagaagcagtgtccccagaacagcggatgcttcagacacctggatgagagggaggagtgcaagtgcctgctgaactacaagcaggaaggagataagtgtgtggaaaaccccaatcctacttgtaacgagaacaatggaggatgcgatgccgatgccaagtgtaccgaggaggattcaggaagcaacggaaagaagatcacctgcgagtgtaccaagcctgattcttatccactgttcgatggtattttctgcagtcaccaccaccaccaccactaactcga